(19) United States
(12) Patent Application Publication
(10) Pub. No.: US 2008/0213332 A1
(43) Pub. Date: Sep. 4, 2008

(54) Cells Isolated from Placenta, Device for Isolating Same, and Uses Thereof

(76) Inventors: Shimon Slavin, Jerusalem (IL); Ido J. Kilemnik, Herzlia (IL)

Correspondence Address: Martin D. Moynihan;PRTSI, P.O.Box 16446, Arlington, VA, 22215, US

(21) Appl. No.: 11/667,492

(22) PCT Filed: Nov. 10, 2005
(86) PCT No.: PCT/IL2005/001184

§ 371 Date: Nov. 13, 2007

Related U.S. Application Data
(60) Provisional application No. 60/626,458, filed on Nov. 10, 2004.

(51) Int. Cl. A61F 2/00; C12N 5/06; C12M 1/00; A61K 35/32; A61P 1/00; A61K 35/36; A61K 35/34; A61K 35/12

(52) U.S. Cl. 424/423; 435/379; 435/381; 435/374; 435/284.1; 435/377; 424/93.7; 424/548; 424/549; 424/574

(57) ABSTRACT

A method of processing an organ is disclosed. The method comprises: (a) placing an organ in a sealable container; (b) disrupting the structure of said organ to yield a cell suspension; and (c) transferring said cell suspension to a sealable cell-suspension storage container, thereby isolating cells of said organ, wherein said sealable container, wherein said disrupting and said transferring are all performed substantially in a continuous vessel.

### FIELD AND BACKGROUND OF THE INVENTION

The present invention relates to devices for harvesting stem cells, to medical implants which comprise stem cells and are capable of generating in-vivo cell populations/tissues derived from mesenchymal and/or hematopoietic stem cells, and to methods of using such implants for treating diseases. More particularly, the present invention relates to devices which comprise a single-use component for harvesting placenta/umbilical cord-derived stem cells in a cryogenically storable format under sterile conditions, to medical implants which comprise placenta/umbilical cord-derived stem cells and are capable of generating bone, cartilage, adipose and/or hematopoietic cells/tissues, and to methods of using such medical implants for treating diseases.

Diseases which are amenable to treatment by implantation of cell populations/tissues derived from mesenchymal and/or hematopoietic stem cells—such as bone, cartilage, adipose tissue and/or hematopoietic cells/tissues—include a vast number of highly debilitating and/or lethal diseases for which no satisfactory/optimal treatment methods are available. Diseases which are amenable to treatment by administration of such cells/tissues include those requiring generation/repair of cells/tissues/organs derived from MSCs/HSCs, and/or those requiring therapeutic immune modulation. Diseases requiring generation/repair of cells/tissues/organs derived from MSCs/HSCs include, for example, cartilage/bone injury, myocardial infarct, and myeloablation following cancer treatment; and diseases requiring therapeutic immune modulation include, for example, transplantation-related diseases, tumors/cancers, autoimmune diseases and infectious diseases.

Mesenchymal stem cells (MSCs) have the capacity to self-renew and differentiate into various lineages of mesenchymal tissues including cortical and trabecular bone, tendons, ligaments and different kinds of cartilage, as well as stromal microenvironment capable of supporting and controlling hematopoiesis (hematopoietic microenvironment) [1-6].

Moreover, cell populations playing an important role in immune regulation, including induction of self tolerance, control of autoimmunity, induction of transplantation tolerance to bone marrow and organ allografts and also possibly controlling graft-versus-host disease following allogeneic stem cell transplantation originate from a common type of early mesenchymal progenitor cells [7; 8]. Because of these features, MSCs may be applied therapeutically for multiple clinical indications, including: 1) treatment of disorders of mesenchymal origin; 2) cell-based therapy of malignant and non-malignant disorders, including autoimmune and other immunological indications and mostly complications of bone marrow transplantation; 3) facilitation of engraftment of bone marrow cells and induction of unresponsiveness to organ allografts; 4) all indications associated with tissue repair and stem cell plasticity.

Prior art methods of using MSCs for disease treatment involve use of adult-stage bone marrow as a stem cell source (Gurevitch et al., 2003. Stem Cells 21:588-597; and U.S. Pat. Nos. 6,752,831, 6,437,018, 5,510,396, 5,507,813, 5,439,684, 5,314,476, 5,298,254 and 5,284,655).

The approach of obtaining MSCs from bone marrow is highly disadvantageous, for example due to the fact that obtaining bone marrow, such as via aspiration from the iliac crest is a highly invasive, painful, cumbersome and expensive procedure. Similarly, obtaining bone marrow cells from the blood of donors is also invasive, cumbersome and expensive, as well as inefficient. Moreover, the prior art use of adult-stage bone marrow as tissue source of stem cells is further associated with the disadvantage that such adult-stage tissues contain cells having a more limited proliferation/differentiation potential, as well as greater immunogenicity for purposes of donor-to-recipient transplantation, relative to tissues at early developmental stages. Furthermore, the bone marrow of cancer patients, which often critically require hematopoietic reconstitution via stem cell administration, is highly unsuitable as a source of stem cells due to contamination, or potential contamination, with malignant cells, even though it theoretically represents an ideal, immunologically matched, stem cell source for such patients. Furthermore, the bone marrow of cancer patients, which often critically require hematopoietic reconstitution via stem cell administration, is highly unsuitable as a source of stem cells due to contamination, or potential contamination, with malignant cells, even though it theoretically represents an ideal, immunologically matched, stem cell source for such patients.

A theoretically optimal strategy for overcoming the limitations of using bone marrow as source of stem cells involves the use of placenta/umbilical cord as a source of stem cells. The placenta/umbilical cord is available for each individual at birth at which time cells isolated therefrom can be cryogenically stored indefinitely for future use during the life of the individual or for transplantation to a recipient. Additionally, the placenta/umbilical cord is at the neonatal stage of development and hence contains cells having greater proliferation/differentiative potential for purposes of regenerative therapy, as well as reduced immunogenicity for purposes of donor-to-recipient transplantation, relative to adult-stage stem cell sources such as bone marrow. Furthermore, placental/umbilical cord cells of an individual destined to be afflicted with cancer later during his/her lifetime are still at a stage during which these will usually be free of the malignant cells which will arise during the lifetime of the individual—in this case placenta/umbilical cord represents a unique and ideal source of perfectly immunologically matched and cancer-free stem cells for hematopoietic reconstitution of the individual following bone marrow-damaging cancer treatment thereof.

The prior art, however, fails to provide a satisfactory/optimal method of obtaining stem cells, such as MSCs and HSCs, and of using such stem cells for disease treatment.

There is thus a widely recognized need for, and it would be highly advantageous to have, a method devoid of the above limitation.

### SUMMARY OF THE INVENTION

The present invention discloses a novel device which can be used for conveniently and routinely obtaining placenta/umbilical cord-derived stem cells from newborns, novel medical implants capable of generating cells/tissues derived from mesenchymal and/or hematopoietic stem cells, and methods of using such implants for treatment of diseases amenable to treatment via administration of such cells/tissues. These uses can be effected in a variety of ways as further described and exemplified hereinbelow.

According to one aspect of the present invention there is provided a method of processing an organ, comprising: (a) placing an organ in a sealable container; (b) disrupting the structure of the organ to yield a cell suspension; and (c) transferring the cell suspension to a sealable cell-suspension storage container, thereby isolating cells of the organ, wherein the disrupting and the transferring are all performed substantially in a continuous vessel.

According to further features in preferred embodiments of the invention described below, the method of processing the organ further comprises: (d) subsequent to (a) and prior to (b), washing the organ.

According to still further features in the described preferred embodiments, the method further comprises: (e) prior to (b), contacting the organ with culture medium.

According to still further features in the described preferred embodiments, the disrupting comprises: (i) physically disrupting the organ to yield organ pieces.

According to still further features in the described preferred embodiments, the disrupting comprises: (ii) digesting connective tissue of the organ to yield the cell suspension.

According to still further features in the described preferred embodiments, the digesting includes adding an enzyme to the organ.

According to still further features in the described preferred embodiments, the method further comprises: (h) adding a cryopreservative to the cell suspension.

According to still further features in the described preferred embodiments, the method further comprises: (j) freezing the cell suspension in the sealable cell-suspension storage container.

According to another aspect of the present invention there is provided a device for processing an organ comprising: (a) an aseptic organ disrupter configured to disrupt an organ into a cell suspension; and (b) a sealable cell-suspension storage container, wherein the aseptic organ disrupter and the cell-suspension storage container constitute a continuous vessel.

According to further features in preferred embodiments of the invention described below, the device further comprises an organ washer configured to wash an organ prior to disruption in the organ disrupter.

According to still further features in the described preferred embodiments, the device further comprises a culture medium inlet functionally associated with the organ disrupter.

According to still further features in the described preferred embodiments, the device further comprises a culture medium reservoir in fluid communication with the organ disrupter through the culture medium inlet.

According to still further features in the described preferred embodiments, the organ disrupter comprises a physical organ disrupter.

According to still further features in the described preferred embodiments, the physical organ disrupter comprises a disrupter component.

According to still further features in the described preferred embodiments, the disrupter component is rotatable.

According to still further features in the described preferred embodiments, the disrupter component is translatable.

According to still further features in the described preferred embodiments, the disrupter component is vibratable.

According to still further features in the described preferred embodiments, the disrupter component includes a sonic transducer.

According to still further features in the described preferred embodiments, the organ disrupter including a connective tissue digester.

According to still further features in the described preferred embodiments, the connective tissue digester includes a digesting liquid inlet.

According to still further features in the described preferred embodiments, the device further comprises a digesting liquid reservoir in fluid communication with the connective tissue digester through the digesting liquid inlet.

According to still further features in the described preferred embodiments, the device further comprises a heater, functionally associated with the connective tissue digester.

According to still further features in the described preferred embodiments, the device further comprises a solid waste separator to separate solid waste from a cell suspension.

According to still further features in the described preferred embodiments, the device further comprises a liquid waste separator to separate liquid waste from a cell suspension.

According to still further features in the described preferred embodiments, the device further comprises an organ holder, substantially a sealable container aseptically reversibly attachable to the organ disrupter.

According to yet another aspect of the present invention there is provided a method of generating a cell population derived from mesenchymal and/or hematopoietic stem cells, the method comprising subjecting to differentiation-inducing conditions cells derived from placenta and/or umbilical cord, the cells derived from placenta and/or umbilical cord being in association with a biocompatible matrix, wherein the differentiation-inducing conditions are selected suitable for inducing differentiation of at least some of the cells derived from placenta and/or umbilical cord into the cell population, thereby generating the cell population.

According to still another aspect of the present invention there is provided a method of treating in a subject a disease amenable to treatment by administration of a cell population derived from mesenchymal and/or hematopoietic stem cells, the method comprising: (a) subjecting to differentiation-inducing conditions cells derived from placenta and/or umbilical cord, the cells derived from placenta and/or umbilical cord being in association with a biocompatible matrix, wherein the differentiation-inducing conditions are selected suitable for inducing differentiation of at least some of the cells derived from placenta and/or umbilical cord into the cell population, thereby generating the cell population; and (b) administering the cell population to the subject, thereby treating the disease in the subject.

According to further features in preferred embodiments of the invention described below, administering the cell population to the subject is effected by administering to the subject an implant which comprises the cells derived from placenta and/or umbilical cord in association with the biocompatible matrix under a renal capsule of the subject.

According to still further features in the described preferred embodiments, the subjecting the cells derived from placenta and/or umbilical cord to the differentiation-inducing conditions is effected by administering to a host which is not the subject an implant which comprises the cells derived from placenta and/or umbilical cord in association with the biocompatible matrix.

According to still further features in the described preferred embodiments, the subjecting the cells derived from placenta and/or umbilical cord to the differentiation-inducing conditions is effected by implanting under a renal capsule of the subject or of a host which is not the subject an implant which comprises the cells derived from placenta and/or umbilical cord in association with the biocompatible matrix.

According to still further features in the described preferred embodiments, the subjecting the cells derived from placenta and/or umbilical cord to the differentiation-inducing conditions is effected for a duration selected from a range of about 30 days to about 150 days.

According to a further aspect of the present invention there is provided a method of treating in a subject a disease amenable to treatment by administration of a cell population derived from mesenchymal and/or hematopoietic stem cells, the method comprising administering to the subject an implant which comprises cells derived from placenta and/or umbilical cord in association with a biocompatible matrix, thereby generating the cell population for treating the disease in the subject.

According to further features in preferred embodiments of the invention described below, administering the implant to the subject is effected by implanting the implant under a renal capsule of the subject.

According to still further features in the described preferred embodiments, the cell population comprises cells selected from the group consisting of osteocytes, chondrocytes, adipocytes and hematopoietic cells, and/or wherein the cell population forms a tissue selected from the group consisting of bone tissue, cartilage tissue, adipose tissue and hematopoietic tissue.

According to yet a further aspect of the present invention there is provided medical implant for treating in a subject a disease amenable treatment by administration of a cell population derived from mesenchymal and/or hematopoietic stem cells, the implant comprising cells derived from placenta and/or umbilical cord in association with a biocompatible matrix.

According to further features in preferred embodiments of the invention described below, the cells derived from placenta and/or umbilical cord are unseparated cells derived from placenta and/or umbilical cord.

According to still further features in the described preferred embodiments, the cells derived from placenta and/or umbilical cord are derived from isolated trophoblast tissue.

According to still further features in the described preferred embodiments, the biocompatible matrix is composed of particles having a minimal diameter of about 310 microns and a maximal diameter of about 450 microns.

According to still further features in the described preferred embodiments, the biocompatible matrix is a demineralized matrix of at least one biological tissue.

According to still further features in the described preferred embodiments, the implant comprises about 1,500,000 of the cells derived from placenta and/or umbilical cord per about 1 milligram of the biocompatible matrix.

The present invention successfully addresses the shortcomings of the presently known configurations by providing a device which can be used for optimally obtaining placenta/umbilical cord-derived stem cells, medical implants capable of generating cells/tissues derived from mesenchymal and/or hematopoietic stem cells, and methods of using such implants for treatment of diseases amenable to treatment via administration of such cells/tissues.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b schematically depict an embodiment of the device of the present invention in cross section.

FIG. 2 schematically depicts an embodiment of the device of the present invention provided with a reversibly aseptically attachable organ holder in cross section.

FIGS. 3a-d are histology photomicrographs depicting generation of compact bone by placental cell-DBM implants. FIG. 3a depicts non-degraded DBM particles 30 days after implantation of DBM alone. FIGS. 3b-d respectively depict clear new bone formation (oppositional osteogenesis) at 30, 60 and 150 days following implantation. Analysis was performed via picroindigocarmin (PIC) staining. Original magnification, ×100.

FIGS. 4a-c are histology photomicrographs depicting the generation of hematopoietic tissue and stromal microenvironment supporting hematopoiesis by placental cell-DBM implants. FIG. 4a depicts that no osteogenesis or hematopoiesis occurs 60 days following implantation of DBM particles alone. FIG. 4b depicts oppositional bone formation and newly developed hematopoietic tissue 30 days following implantation. FIG. 4c depicts newly formed bone trabeculae and completely developed hematopoietic cavity 150 days following implantation. Recipient kidney sections were stained with hematoxylin-eosin (H&E). Original magnification, ×100.

FIGS. 5a-d are histology photomicrographs depicting generation of cartilage and adipose tissue by placental cell-DBM implants. FIG. 5a depicts that no bone tissue, cartilage tissue, adipose or hematopoietic tissues are generated 150 days after transplantation of DBM particles alone. FIGS. 5b-c respectively depict development of cartilage at 30 and 150 days after implantation. FIG. 5d depicts clearly visible adipose tissue formed in developing bone marrow cavity 60 days following implantation. Analysis was performed via picroindigocarmin (PIC) staining. Original magnification, ×100.

FIG. 6a is a series of photomicrographs depicting mineral deposition in unseparated umbilical cord cells cultured under differentiation-inducing conditions for 18 and 49 days, as determined via alizarin red S staining.

FIG. 6b is a series of photomicrographs depicting osteogenic differentiation in unseparated umbilical cord cells cultured under osteogenic differentiation-inducing conditions, as determined via alkaline phosphatase staining.

FIG. 7a depicts osteogenic differentiation in unseparated mouse umbilical cord cells. cultured under osteogenic differentiation-inducing conditions. Cells were cultured for 24, 28 or 31 days and stained with NBT or Alizarin red S.

FIG. 7b depicts osteogenic differentiation in unseparated mouse umbilical cord cells. cultured under osteogenic differentiation-inducing conditions with bFGF treatment. Cells were cultured for 24, 28 or 31 days with or without bFGF treatment, and stained with NBT or Alizarin red S.

FIG. 8a-b depict chondrogenesis and osteogenesis, respectively, in unseparated mouse umbilical cord cells implanted with demineralized bone matrix under the renal capsule of mouse recipients.

FIG. 9 is a bar-graph depicting strong immunosuppression of allogeneic mixed lymphocyte reaction by trophoblast cells and umbilical cord cells. Trophoblast or umbilical cord cultured cells irradiated at 1,500 cGy were added to MLR cultures employing Balb/c stimulators irradiated at 5,000 cGy and C57BL/6 allogeneic responders.

FIGS. 10a-g are photomicrographs depicting chondrogenesis, osteogenesis and hematopoietic marrow formation by mouse trophoblast cells implanted with trophoblast cells and demineralized bone matrix under the renal capsule of a mouse recipient. Two-million trophoblast cells were implanted. FIG. 10a depicts primary bone deposited on hyalin cartilage. FIG. 10b depicts primary bone with adjacent hematopoietic marrow. FIG. 10c depicts trabecular bone with red and yellow bone marrow. FIGS. 10d-g respectively depict generation of hyalin cartilage, hematopoietic stroma, hematopoiesis and yellow bone marrow at high resolution.

### DESCRIPTION OF THE PREFERRED EMBODIMENTS

The present invention is of a method of obtaining cells from an organ, of a device for practicing the method, of a medical implant which comprises placenta/umbilical cord-derived cells and is capable of generating in-vivo cells and tissues derived from mesenchymal and/or hematopoietic stem cells, and of a method of using such an implant for treating diseases. Specifically, the present invention can be used to obtain mesenchymal and/or hematopoietic stem cells from placenta/umbilical cord in cryogenically storable format conveniently, economically and effectively, and can be used for routine and effective treatment of diseases which are amenable to treatment by implantation of cells/tissues which are derived from such stem cells, such as cartilage, bone, adipose and/or hematopoietic cells/tissues.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. It is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Diseases amenable to treatment via administration of cells/tissues derived from mesenchymal stem cells (HSCs) and/or hematopoietic stem cells (HSCs) include a vast number of highly debilitating and/or lethal diseases for which no satisfactory/optimal therapy exists. However, the prior art approaches for practicing such disease treatment involve isolation of stem cells from adult-stage bone marrow, a procedure which is disadvantageous due to being highly invasive, cumbersome expensive and/or inefficient to practice, and hence essentially impossible to routinely practice according to need. Moreover, the prior art use of adult-stage bone marrow-derived stem cells is further associated with the disadvantage that such adult-stage tissues contain cells having a more limited proliferation/differentiation potential, as well as greater immunogenicity for purposes of donor-to-recipient transplantation, relative to tissues at earlier developmental stages. Furthermore, the bone marrow of cancer patients, which often critically require hematopoietic reconstitution via stem cell administration following bone marrow-damaging cancer treatment, is highly unsuitable as a source of stem cells due to contamination, or potential contamination, with malignant cells, even though it theoretically represents an ideal, immunologically matched, stem cell source for such patients.

A theoretically optimal strategy for overcoming the limitations of using bone marrow as source of stem cells involves the use of placenta/umbilical cord as a source of stem cells. The placenta/umbilical cord is available for each individual at birth at which time cells isolated therefrom can be cryogenically stored indefinitely for future therapeutic administration to the individual or to a recipient. Additionally, the placenta/umbilical cord is at the neonatal stage of development and hence contains cells having greater proliferation/differentiative potential for purposes of regenerative therapy, as well as reduced immunogenicity for purposes of donor-to-recipient transplantation, relative to adult-stage stem cell sources such as bone marrow. Furthermore, placental/umbilical cord-derived cells of an individual destined to be afflicted with cancer later during his/her lifetime are still at a stage during which these will usually be free of the malignant cells which will arise during the lifetime of the individual—in this case placenta/umbilical cord represents a unique and ideal source of perfectly immunologically matched and cancer-free stem cells for hematopoietic reconstitution of the individual following bone marrow-damaging cancer treatment thereof.

The prior art; however, fails to provide a satisfactory/optimal method of obtaining stem cells, such as MSCs and HSCs, and of using such stem cells for disease treatment.

While reducing the present invention to practice, as described in the Examples section which follows, the present inventors have uncovered that implantation into a host mammal of an implant of unseparated placenta/umbilical cord cells associated with particles of demineralized bone matrix (DBM) can be used to routinely and conveniently generate in the host mammal cells/tissues derived from MSCs/HSCs, such as bone, cartilage, adipose tissue and hematopoietic stroma capable of supporting and controlling hematopoiesis. Hence, the present inventors have uncovered while reducing the present invention to practice that such implants can be used to treat diseases which are amenable to treatment by administration of such cells/tissues.

In order to overcome the prior art limitations associated with use of adult-stage bone marrow as a source stem cells for disease treatment, and in view of the fact that placenta/umbilical cord represents an ideal source of stem cells, such as MSCs and HSCs, the present inventors have conceived a device for preparation and cryopreservation/freezing of isolated placenta/umbilical cord cells.

Thus, according to the teachings of the present invention there is provided a method of processing an organ (e.g. an after-birth including a placenta and/or umbilical cord), comprising: a) placing an organ in an aseptic container; b) disaggregating the organ to yield an organ disaggregate; and c) transferring the organ disaggregate to a sealable organ disaggregate storage container, thereby isolating cells of the organ, wherein the disaggregating and the transferring are all performed substantially in a continuous vessel. In other words, from the moment the organ (preferably substantially whole and undamaged) is placed in the aseptic container it is held and processed within a continuous aseptic vessel to yield an organ disaggregate. In preferred embodiments, the organ is an after-birth, a placenta and/or an umbilical cord.

In preferred embodiments, the organ disaggregate is a cell suspension. When the organ is an after-birth, a placenta and/or an umbilical cord, such a cell suspension generally includes suspended stem cells, such as mesenchymal and/or hematopoietic stem cells

In embodiments of the present invention, subsequent to placing the organ in the aseptic container but prior to disaggregating the organ, the organ is washed.

Preferably the disaggregation of the organ occurs within a culture medium. Therefore, in embodiments of the present invention, prior to disaggregating the organ, the organ is contacted with culture medium, preferably is substantially immersed in culture medium.

Many methods of disaggregation are known in the art (see for example Freshney, Culture of Animal Cells, A Manual of Basic Technique, 2d Ed., A. R. Liss, Inc., New York, 1987, Ch. 9, pp. 107-126) and can be adapted for use in implementing the teachings of the present invention.

In embodiments of the present invention, disaggregating the organ comprises i) physically disaggregating the organ to yield organ pieces. In embodiments of the present invention, physically disaggregating includes at least one step or process selected from the group consisting of blending, braying, chopping, comminuting, crushing, cubing, cutting, disintegrating, disrupting, grinding, liquefying, mashing, mincing, mushing, pressing, shredding, smashing, squashing, squeezing, squishing, teasing, mincing, slicing and dicing the organ.

In embodiments of the present invention, disaggregating the organ comprises ii) digesting connective tissue (e.g. extracellular matrix) of the organ to yield an organ disaggregate, for example by adding an enzyme (e.g. trypsin, chymotrypsin, collagenase, elastase, hyaluronidase, DNase or a combination thereof) to the organ. Disaggregating the organ is preferably performed

In embodiments of the present invention, i) physical disaggregation and ii) digesting connective tissue are used together to disaggregate the organ to yield an organ disaggregate. The physical disaggregation disrupts tenacious connective tissue such as tough external membranes and yields organ pieces with a high total surface area allowing the digesting to occur efficiently. The digesting digests extracellular matrix and other intercellular connective tissue, separating cells from each other to yield the desired organ disaggregate.

In embodiments of the present invention, subsequent to the disaggregation of the organ structure, liquid is removed from the organ disaggregate, before or after transfer of the organ disaggregate to the sealable organ disaggregate storage container. to Such liquid includes liquid released from the organ as well as excess culture medium.

In embodiments of the present invention, subsequent to the disaggregation of the organ, non-cell solids are removed from the organ disaggregate, before or after transfer of the organ disaggregate to the sealable organ disaggregate storage container.

Such non-cell solids includes pieces of connective tissue and the like and generally includes some cells.

In embodiments of the present invention, a cryopreservative (e.g. dimethylsulfoxide) is added to the organ disaggregate. A cryopreservative is added to the organ disaggregate at any time during performance of the method of the present invention, e.g. prior to disaggregation of the organ structure, prior to transfer of the organ disaggregate to the sealable organ disaggregate storage container or subsequently to transfer of the organ disaggregate to the sealable organ disaggregate storage container.

In embodiments of the present invention, the organ disaggregate (preferably with added cryopreservative) is frozen in the sealable organ disaggregate storage container.

According to the teachings of the present invention, there is also provided a device for processing an organ comprising: a) an aseptic organ disaggregator configured to disaggregate the organ into an organ disaggregate; and b) a sealable organ disaggregate storage container (preferably cryogenically storable), wherein the aseptic organ disaggregator and the organ disaggregate storage container constitute a continuous vessel.

In embodiments of the present invention, the device further comprises an organ washer configured to wash the organ prior to disaggregation. In embodiments of the present invention, the organ washer includes a wash liquid inlet, preferably comprising a wash liquid valve, preferably a unidirectional valve. In embodiments of the present invention, a wash liquid source such as a faucet or wash liquid reservoir such as a bag of sterile wash liquid is in fluid communication to the wash inlet. In embodiments of the present invention a wash liquid reservoir is a fixed component of the device

In embodiments of the present invention, the device further includes a wash liquid drain in fluid communication with the organ washer, preferably comprising a drain valve, preferably a unidirectional drain valve. In embodiments of the present to invention, the liquid drain is in fluid communication with a wash liquid waste container. In embodiments of the present invention, the device further comprises a suction component functionally associated with the wash liquid drain (e.g. directly or through a wash liquid waste container) allowing efficient removal of wash liquid.

In embodiments of the present invention, the device further comprises a positive pressure generator functionally associated with the organ washer. When activated, the positive pressure generator compresses a fluid (such as air or a gas) into the organ washer so as to force wash liquid out of the organ washer through the wash liquid drain.

In embodiments of the present invention, the device further comprises a culture medium inlet functionally associated with the organ disaggregator, allowing addition of culture medium before, during or after disaggregation of the organ structure so as to increase cell viability. A culture medium inlet preferably comprises a culture medium valve, preferably a unidirectional culture medium valve. In embodiments of the present invention, a culture medium source such as a culture medium reservoir such as a bag of sterile culture medium is in fluid communication with the culture medium inlet. In embodiments of the present invention a culture medium reservoir is a fixed component of the device

In embodiments of the present invention, the organ disaggregator includes a physical organ disaggregator, generally provided with a disaggregation component. Disaggregation components include rotatable disaggregation components such as rotating blades, blender blades and stirrers, translatable disaggregation components such as ricers and dicers, vibratable disaggregation components such as vibrating blades, or sonic (e.g. ultrasonic) transducers for physically disaggregating the organ with sonic vibrations, or combination of rotatable, translatable, vibrating or sonic disaggregation components.

In embodiments of the present invention, a disaggregation component is mechanically driven, that is that motion is mechanically transferred to a moving disaggregation component by a mechanical drive.

In embodiments of the present invention, a disaggregation component is non-mechanically driven, that is that motion is transferred to a moving disaggregation component not mechanically. Typical non-mechanical drives include coupled magnets (magnetic stirrers and the like). A non-mechanical drive avoids penetration of a wall of the device In embodiments of the present invention a disaggregation component includes a unit selected from the group consisting of a blender, a brayer, a chopper, a comminuter, a crusher, a cuber, a cutter, a disintegrator, a disrupter, a grinder, a liquidizer, a masher, a mincer, a musher, a press, a rotor, a smasher, a squasher, a squeezer, a teaser, a shredder, a ricer, a slicer and a dicer.

In embodiments of the present invention, the device further comprises a positive pressure generator functionally associated with the physical organ disaggregator. When activated, the positive pressure generator compresses a fluid (such as air or a gas) into the physical organ disaggregator so as to force the pieces of organ formed by the action of the physical organ disaggregator out of the physical organ disaggregator for further processing.

In embodiments of the present invention, the organ disaggregator includes a connective tissue digester. In the connective tissue digester, the cells of the organ are separated one from the other and preferably from connective tissue and the like, so that ultimately the organ is processed into a suspension including single cells suspended in a liquid. In embodiments of the present invention, the connective tissue digester includes a digesting liquid inlet. In embodiments of the present invention the digesting liquid inlet comprises a diaphragm allowing injection of digesting liquid therethrough.

In embodiments of the present invention, the digesting liquid inlet comprises a digesting liquid valve. In embodiments of the present invention, the device further comprises a digesting liquid reservoir in fluid communication with the connective tissue digester through the digesting liquid valve. In embodiments of the present invention a digesting liquid reservoir is a fixed component of the device. In embodiments of the present invention, the device comprises an enzyme solution (e.g. trypsin, chymotrypsin, collagenase, elastase, hyaluronidase, DNase or a combination thereof) contained in the digesting liquid reservoir. Guidance regarding suitable enzymatic cellular disagreggation of a placenta/umbilical cord is available in the literature of the art (refer, for example, to Zhang Y. et al., 2004. Chin Med J (Engl). 117:882-887)

In embodiments of the present invention, a device of the present invention further comprises a heater functionally associated with the connective tissue digester. In embodiments of the present invention, the heater is configured to heat the contents of the connective tissue digester directly, for example by heating the walls of the connective tissue digester. In embodiments of the present invention, the heater is configured to heat the contents of the connective tissue digester indirectly, for example, by heating the digesting liquid or culture medium before addition to the organ disaggregator.

In embodiments of the present invention, the device further comprises a positive pressure generator functionally associated with the connective tissue digester. When activated the positive pressure generator compresses a fluid (such as air or a gas) into the connective tissue digester so as to force the organ disaggregate formed by the action of the connective tissue digester out of the connective tissue digester for further processing.

In embodiments of the present invention, the device further comprises a solid waste separator to separate solid waste from an organ disaggregate. In embodiments of the present invention, the device further comprises a liquid waste separator to separate liquid waste from an organ disaggregate. In embodiments of the present invention, the organ disaggregate storage container is substantially a prior art cryopreservation bag provided with a main bag and one or more waste bags. Once such a bag is sealed, solid and liquid waste is separated from the desired organ disaggregate in the usual way with which one skilled in the art is familiar.

In embodiments of the present invention, the device further comprises a cryopreservative liquid inlet. In, embodiments provided with a cryopreservative liquid valve, preferably a unidirectional valve. In embodiments of the present invention, a cryopreservative liquid reservoir is in fluid communication with the organ disaggregate storage container through the cryopreservative liquid inlet.

In embodiments of the present invention, the device further comprises a sterilizer functionally associated with components of the continuous vessel.

In embodiments of the present invention, the sterilizer emits sterilizing radiation, e.g. ultraviolet, infra red, microwave or gamma radiation.

In embodiments of the present invention the sterilizer injects a sterilizing liquid into components of the continuous vessel, e.g. concentrated salt solutions, concentrated sugar solutions or formaldehyde solutions.

In embodiments of the present invention, the sterilizer injects a sterilizing gas into components of the continuous vessel, e.g. steam, chlorine or ethylene oxide.

In embodiments of the present invention, one or more components described above as separate components are combined into a single component having one or more functions.

In embodiments of the present invention, the disaggregation component of the physical organ disaggregator is located in the component that is also the organ washer. Subsequently to washing of the organ, the disaggregation component is activated. In such embodiments, it is often advantageous to combine a wash liquid inlet and a culture medium inlet to one component and even to combine a wash liquid reservoir and a culture medium reservoir to one component.

In embodiments of the present invention, the organ disaggregator is substantially a single component that is both a physical organ disaggregator and a connective tissue digester. In such embodiments, physical disaggregation and connective tissue digestion are preferably performed substantially simultaneously.

In embodiments of the present invention, the organ washer, the physical organ disaggregator and the connective tissue digester are all substantially a single component.

In embodiments of the present invention, the organ washer is a component physically separated from the connective tissue digester but in fluid communication therewith through a physical organ disaggregator. In such embodiments, once an organ is washed in an organ washer, the organ is transferred to the connective tissue digester while being physically disrupted by the physical organ disaggregator.

In embodiments of the present invention, the device is substantially an integral unit having a lid to allow placement of an organ within.

In embodiments of the present invention, a device is substantially a self-contained integral unit with only few optional connectors including a drive, power supply or control unit for a physical organ disaggregator, or to a wash liquid source (unless provided with a wash liquid reservoir as a fixed component), to wash drain (unless provided with a wash liquid waste container as a fixed component), one or more positive pressure generator inlets, one or more vacuum ports, a culture medium source (unless provided with a culture medium reservoir as a fixed component), a digesting liquid source (unless provided with a digesting liquid reservoir as a fixed component), a cryopreservative source (unless provided with a cryopreservative reservoir as a fixed component), a heater power supply and inlet-ports and/or power supplies for a sterilizer.

In embodiments of the present invention, the device further comprises an organ holder, substantially a container aseptically reversibly attachable to the organ disaggregator. Once attached to the organ disaggregator, an organ held in an organ holder can be aseptically transferred to the organ disaggregator.

An additional aspect of the present invention is a method and a device for processing an organ to provide an organ disaggregate, for example an organ that has been surgically removed or has been expelled from the body, for example an after-birth. The teachings of the present invention allow for simple processing of an organ to yield a storable organ disaggregate with little intervention of medical personnel. An organ disaggregate made in accordance with the teachings of the present invention is aseptic and thus suitable for use in the preparation of medicaments and can be cryogenically stored despite originating from the relatively unclean environment from which the organ is harvested.

The present invention is suitable for processing of large pieces of tissue, especially organs such as brains, kidneys, glands, liver, lungs, hearts, ovaries, testes and pancreas. The present invention is especially useful for processing the after-birth including a placenta/umbilical cord to provide a storable organ disaggregate including stem cells from the placenta/umbilical cord, e.g. mesenchymal stem cells and hematopoietic stem cells. When necessary to use the stem cells for, e.g. therapeutic purposes, the disaggregate is recovered and processed in the usual way.

In general, according to the method of the present invention an organ is placed in aseptic container. Preferably the organ is placed whole and undamaged so that the natural protective structure of the organ are uncompromised. Once inside the aseptic container, and thus protected from contamination, the organ is disaggregated to yield the desired organ disaggregate. The organ disaggregate is subsequently stored. A feature of the present invention is that the steps of the method, subsequent to placing the organ in the aseptic container are all performed aseptically substantially in a single continuous vessel, with little intervention and preferably, substantially autonomously.

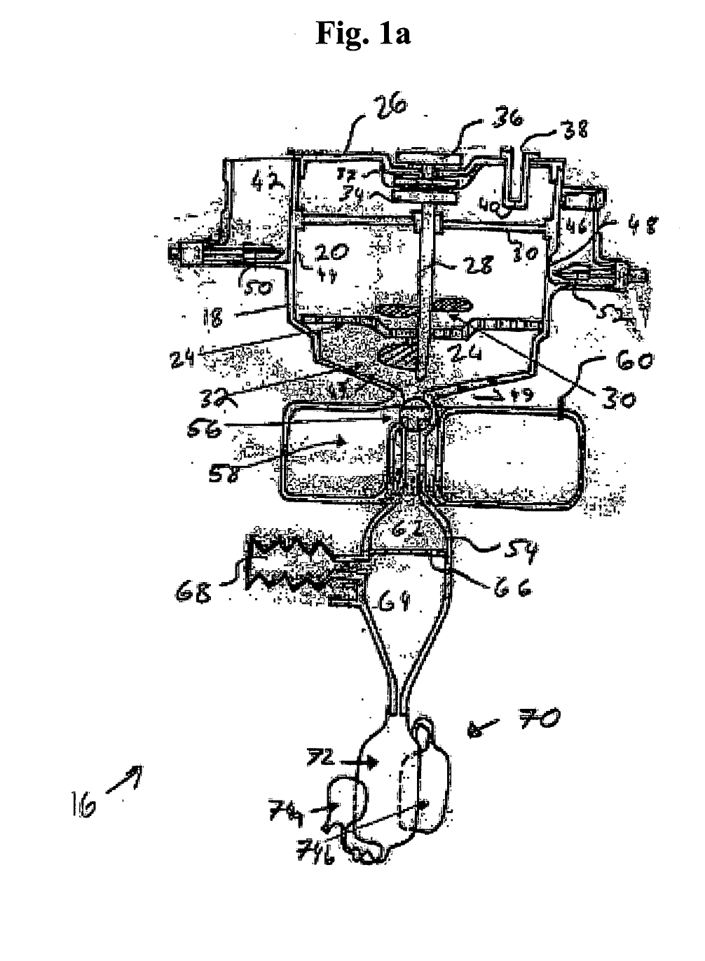

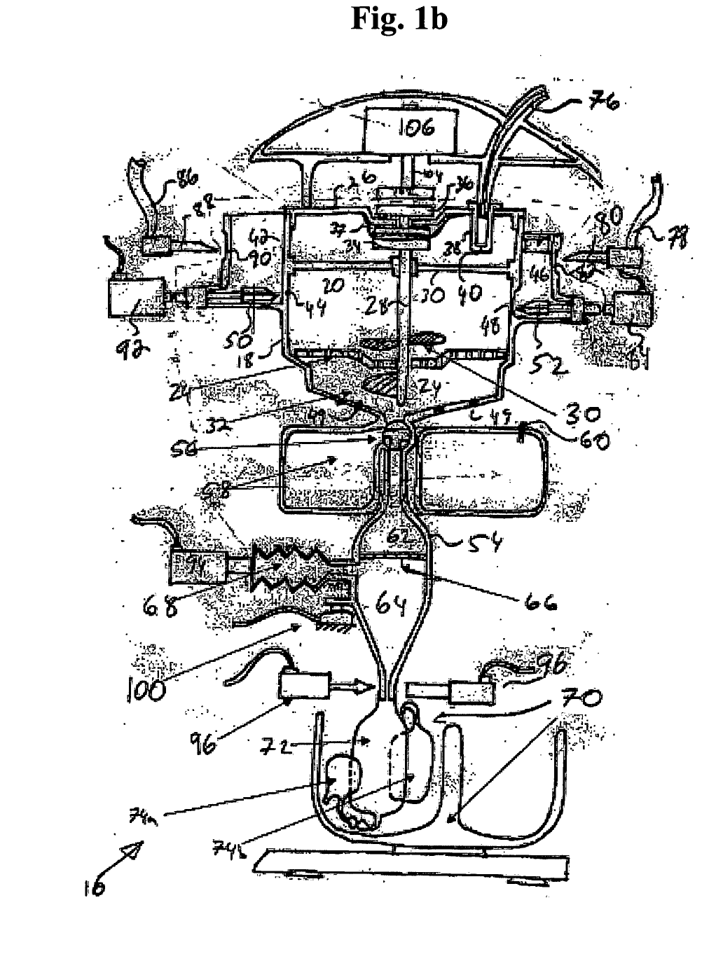

An embodiment of the method of the present invention will be explained with reference to an embodiment of a device 16 of the present invention depicted in FIG. 1A, alone in cross-section, and FIG. 1B, attached to a connector cradle of which various components in cross-section.

Device 16 includes a first chamber 18 divided into an upper part 20 and a lower part 22 by a filter 24 (a nylon mesh with 0.2 mm pores) and is provided with a sealable lid 26. A shaft 28 is rotatably supported substantially vertically by struts 30. Attached to shaft 28 above filter 24 is a rotating blade 30 and below filter 24 is a mixing rotor 32. A shaft drive magnet 34 is attached to shaft 28 and is functionally associated with a magnetic drive plate 36. Shaft drive magnet 34 is physically separated from magnetic drive plate 36 by thin partition 37. When magnetic drive plate 36 rotates, the magnetic attraction between shaft drive magnet 34 and magnetic drive plate 36 causes shaft drive magnet 34, and consequently shaft 28, to rotate despite the lack of mechanical connection or physical contact between magnetic drive plate 36 and shaft drive magnet 34. Thin partition 37 prevents contaminants from entering first chamber 18 along the shaft of magnetic drive plate 36.

Associated with first chamber 18 is wash liquid inlet 38 including unidirectional wash liquid valve 40, culture medium chamber 42 separated from first chamber 18 with a diaphragm 44 (together constituting a culture medium inlet) and digesting liquid chamber 46 separated from first chamber 18 with a diaphragm 48 (together constituting a digesting liquid inlet). A culture medium wall piercer 50 opposes diaphragm 44 and a digesting liquid wall piercer 52 opposes diaphragm 48. In the bottom of lower part 22 of first chamber 18 are embedded heating elements 49. Upper chamber 18 together with associated components substantially constitute an organ washer and aseptic organ disaggregator including both a physical organ disaggregator with a rotatable disaggregation component (rotating blade 30) and a connective tissue digester.

Lower part 22 of first chamber 18 is in communication with a second chamber 54 through a T-valve 56 with three-states: a shut state (depicted) preventing fluid communication sealing the bottom of first chamber 18 and second chamber 54, a drain state (rotate 180° from the depicted) allowing fluid communication between first chamber 18 and torus-shaped wash-liquid waste container 58 and a flow state (rotate 90° anti-clockwise from the depicted) allowing fluid communication between first chamber 18 and second chamber 54. T-valve 56 substantially constitutes a wash liquid drain of device 16.

Torus-shaped wash-liquid waste container 58 is provided with a unidirectional air vent 60, configured to allow air to escape from waste container 58 when liquid enters waste container 58.

Second chamber 54 is divided into an upper part 62 and a lower part 64 by a filter 66 (a nylon mesh with 0.5 mm pores). Entering just below filter 66 is air-inlet 68 for cleaning filter 66 if plugged by back-blowing. Lower part 64 of second chamber 54 is in fluid communication with a cryogenically storable organ disaggregate storage container 70, substantially a prior art cryopreservation bag provided with a main bag 72 and two auxiliary bags 74a and 74b. As is explained below, organ disaggregate storage container 70 functions as a solid waste separator and as a liquid waste separator.

Device 16 constitutes a continuous vessel from first chamber 18 through T-valve 56 through second chamber 54 to organ disaggregate storage container 70.

In FIG. 1B, device 16 is depicted attached to a connector cradle. A power supply (not depicted) is attached to heating elements 49. A wash liquid supply 76 is connected to wash liquid inlet 38. A digesting liquid supply 78 associated with a digesting liquid inlet piercer 80 opposes a diaphragm 82 of digesting liquid chamber 46. A solenoid 84 is positioned to push digesting liquid piercer 52. A culture medium inlet 86 associated with a culture medium inlet piercer 88 opposes a diaphragm 90 of culture medium chamber 42. A solenoid 92 is positioned to push culture medium piercer 50. An air source 94 is attached to air inlet 68. A sealing device 96 is positioned relative to the neck of organ disaggregate storage container 70 to allow sealing of organ disaggregate storage container 70 when desired. Organ disaggregate storage container 70 is supported by a storage container holder 98. Magnetic drive plate 36 physically engages motor drive plate 102 that is attached to shaft 104 of motor 106. A weighing component 100 is positioned to weigh device 16. Since the weight of device 16 is known, the weight of an organ held in device 16 is easily calculated.

The method of the present invention and use of the device of the present invention is described with reference to device 16 depicted in FIGS. 1a-b.

After an after-birth is expelled by a mother, the after-birth is placed (with or without collection of cord blood) inside first chamber 18. Lid 26 is shut, sealing first chamber 18.

Device 16 is attached to the connector cradle and the various connectors and inlets attached.

Wash liquid is supplied from wash liquid supply 76 through wash liquid inlet 38 and wash liquid valve 40 to wash dirt, blood and contamination from the after-birth. T-valve 56 is set to the drain state so that contaminated wash liquid drains away into torus-shaped wash-liquid waste container 58. Suitable wash liquids include water, physiological fluids and even cell culture medium.

When the after-birth is sufficiently washed, T-valve 56 is set to a shut state.

Solenoid 92 is activated, pushing culture medium wall piercer 50 through diaphragm 44 so that culture chamber 42 is in fluid communication with first chamber 18. Culture medium inlet piercer 88 is pressed through diaphragm 90. Culture medium is supplied through culture medium supply 86, flows through culture medium chamber 42 and then into first chamber 18. The purpose of culture medium is to provide bulk and reduce viscosity during the organ disaggregation process. In principle, any liquid that does not compromise the viability of cells released from an organ is suitable for use as a culture medium in implementing the teachings of the present invention. Suitable culture medium includes any culture medium with which one of average skilled in the art is acquainted. Preferably the used culture medium is formulated to avoid inducing differentiation of stem cells present in placental/umbilical cord tissue. The amount of culture medium used is dependent on the geometry of first chamber 18.

Solenoid 84 is activated, pushing digesting liquid wall piercer 52 through diaphragm 48 so that digesting liquid chamber 46 is in fluid communication with first chamber 18. Digesting liquid inlet piercer 80 is pressed through diaphragm 82. Digesting liquid is supplied through digesting liquid supply 78, flows through digesting liquid chamber 46 and then into first chamber 18. Suitable digesting liquids are digesting liquids known for attacking, digesting or disintegrating extracellular matrix and other intercellular connective material, so as to separate cells making up the after-birth from each other. Suitable digesting liquids include solutions of enzymes or chelating agents. Suitable enzymes include trypsin, chymotrypsin, collagenase, elastase, hyaluronidase or combinations thereof. The appropriate amount of a given digesting liquid is calculated by one of average skill in the art without undue experimentation upon perusal of the description herein.

During and after addition of culture medium and digesting liquid, motor 106 is activated, rotating motor drive plate 102. Rotation of motor drive plate 102 rotates magnetic drive plate 36. Rotation of magnetic drive plate 36 induced shaft drive magnet 34 to rotate, causing shaft 28, rotating blade 30 and mixing rotor 32 to rotate.

The action of rotating blade 30 physically disrupts the structural integrity of the after-birth and of tenacious membranes of the after-birth. The after-birth is physically reduced to smaller and smaller pieces and thus acquires an increasingly large surface area. The large surface area of the physically disrupted after-birth allows effective digestion of the intracellular matrix and other connective tissue by the digesting liquid holding the cells of the after-birth together. The mixture is heated by the action of heating elements 49 to an optimum rate of digestion by the digesting liquid. The action of mixing rotor 32 ensures continuous mixing of the mixture, and passage of the mixture between upper part 20 and lower part 22 of first chamber 18. With time, the after-birth substantially becomes an organ disaggregate with floating bits of undigested connective tissue and the like.

When sufficient time has passed, T-valve 56 is set to the flow state, allowing the organ disaggregate to drain from first chamber 18 into second chamber 54 and from second chamber 54 into organ disaggregate storage container 70. Larger fragments of after-birth do not pass through filter 24 and filter 66.

When sufficient organ disaggregate has drained into organ disaggregate storage container 70, sealing device 96 is actuated to seal the gathered organ disaggregate inside organ disaggregate storage container 70.

In the manner known to one skilled in the art, substantially non-cellular solid matter that settles at the bottom of main bag 72 of organ disaggregate storage container 70 is transferred to auxiliary bag 74a and discarded. Main bag 72 is then centrifuged to provide a cell-rich substance (including both mesenchymal and/or hematopoietic stem cells) that is transferred to auxiliary bag 74b while the supernatant is discarded in main bag 72. A cryopreservative (e.g. 10 percent dimethylsulfoxide with 4 percent human serum albumin in saline) is added to the cell-rich substance in auxiliary bag 74b for storage, for example at minus 196 degrees centigrade, in the usual way.

Device 16 is designed to be substantially entirely disposable after one use. Thus, device 16 is detached from the connector cradle and, together with biological waste captured on filters 24 and 66, and waste liquid held in wash liquid waste container 58, discarded.

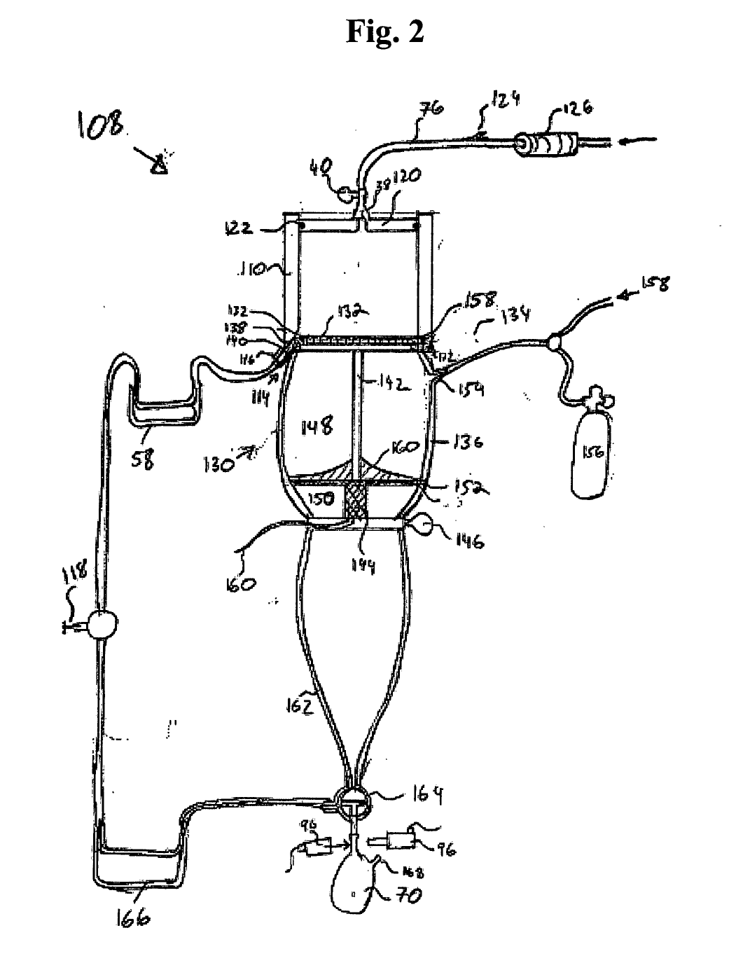

An additional embodiment of a device of the present invention 108 is depicted in FIG. 2. Device 108 is different from device 16 in that device 108 is intended to be reusable. Device 108 is provided with an organ holder 110 that is aseptically reversibly attachable to the other components of device 108 through collar 112. Organ holder 110 is designed to be disposable. In the vicinity of collar 112 is found wash liquid drain 114 comprising a unidirectional drain valve 116. Drain valve 116 is attached to a suction component 118 (a vacuum pump) through a wash liquid waste container 58.

Organ holder 110 is a parallel-walled container with a tight-fitting lid 120 provided with a seal 122 so that lid 120 is configured to slide downwards, while maintaining sealing, into organ holder 110 upon application of sufficient pressure from above. Lid 120 is also provided with a wash liquid inlet 38 provided with a unidirectional wash liquid valve 40. Wash liquid inlet 38 and wash liquid valve 40 configure organ holder 110 to function as an organ washer.

Wash liquid inlet 38 is connected with wash liquid supply 76 which, as is discussed below, is used to provide wash liquid, culture medium, and digesting liquid to device 108. Wash liquid supply 76 is provided with a digesting liquid inlet diaphragm 124 and with a liquid heating element 126.

Collar 112 of organ holder 110 is configured, while maintaining sealing, to engage neck 128 of organ disaggregator 130 which is provided with iris valve 132. When collar 112 engages neck 128 and iris valve 132 is open, there is an aseptic passage between organ holder 110 and organ disaggregator 130.

Wash liquid inlet 38 is configured to function also as a culture medium inlet, to allow the addition of culture medium to organ disaggregator 130 when iris valve 132 is open. Consequently, wash liquid valve 40 is also considered to be a culture medium valve.

Wash liquid inlet 38 is configured to function also as a digesting liquid inlet, to allow the addition of digesting liquid to organ disaggregator 130 when iris valve 132 is open. Consequently, wash liquid valve 40 is also considered to be a digesting liquid valve.

Organ disaggregator 130 is divided into two main sections, a substantially ring-shaped physical organ disaggregator 134 and a vase-shaped connective tissue digester 136, where organ holder 110 is in fluid communication with connective tissue digester 136 through tubular physical organ disaggregator 134. Organ disaggregator 130 is configured to disaggregate the structure of an organ passing first through physical organ disaggregator 134 and then into connective tissue digester 136 into an organ disaggregate.

Physical organ disaggregator 134 is considered to begin from iris valve 132. Flush with the downstream side of iris valve 132 are two sets of parallel knives 138 (constituting a static disaggregation component), the sets arrayed perpendicularly in the fashion of an onion chopper or a chips maker, followed by a slicing disk 140 (constituting a rotatable disaggregation component) mounted on a shaft 142 attached to an electric motor 144.

The upstream end of connective tissue digester 136 begins with slicing disk 140 and the downstream end of connective tissue digester 136 ends with valve 146.

Connective tissue digester 136 is divided into an upper chamber 148 and a lower chamber 150 by a filter 152 (a perforated steel plate with 1 mm holes).

Organ disaggregator 130 is also provided with a positive pressure inlet 154. Positive pressure inlet 154 is functionally associated with positive pressure generator 156 and sterilizing gas source 158, in device 108 a steam generator.

Electric motor 144 is mounted in a water-proof case in lower chamber 150 so that the upper end of motor 144, wherefrom shaft 142 emerges, is flush with the top surface of filter 152. Electrical power for motor 144 is transported through wires 160 that emerge through the walls of valve 146.

As noted above, slicing disk 140 is mounted on shaft 142. Further, stirring rotor 160 is also mounted on shaft 142 so that the downstream edges of the vanes of stirring rotor 160, when rotating, substantially scrape over the upstream surface of filter 152. Lower chamber 150 is in fluid communication with solid and liquid waste separator 162 through valve 146.

Waste separator 162 is substantially an inverted bottle shaped vessel terminating downstream at a T-valve 164 with three-states: a shut state (depicted) sealing the bottom of waste separator 162, a drain state (rotate 180° from the depicted) allowing fluid communication between waste separator 162 and suction component 118 through waste container 166 and a flow state (rotate 90° anti-clockwise from the depicted) allowing fluid communication between waste separator 162 and sealable organ disaggregate storage container 70.

Sealable organ disaggregate storage container 70 is a cryogenically storable bag attached to the outlet of waste separator 162. A cryopreservative or the like can be injected into organ disaggregate storage container 70 through cryopreservative inlet 168. The neck of organ disaggregate storage container 70 is situated inside sealing device 96.

Device 108 constitutes a continuous vessel from organ holder 110 through organ disaggregator 130 through waste separator 162 through organ disaggregate storage container 70.

The use of device 108 is substantially similar to that of device 16 with a few notable differences.

Generally, organ holder 110 with matching lid 120 and a removable collar seal (not depicted, to seal collar 112) are in a remote location where birth is given, whether at home, in an ambulance or in a hospital. If desired, organ holder 110 may contain ice or cold culture medium. When born, the after-birth is placed inside organ holder 110 and lid 120 used to shut organ holder 110.

Shut organ holder 110 holding the after-birth is transported to a location where the other components of device 108 are located, for example at a stem cell bank or the like. Any removable collar seal is removed and collar 112 engaged while maintaining a seal with neck 128 of organ disaggregator 130. Suction component 118 is attached to drain valve 116 and wash liquid supply 76 attached to wash liquid valve 40.

Wash fluid is repeatedly introduced into organ holder 110 through wash liquid valve 40 and removed from organ holder 110 into wash liquid waste container 58 using suction component 118 to wash the after-birth. Wash fluid is preferably heated using liquid heating element 126 to a temperature that is appropriate for optimal digestion by the digesting liquid to ensure that the after-birth is sufficiently warm. The wash fluid used is preferably a culture medium.

When the after-birth is sufficiently washed, iris valve 132 and valve 146 are opened and electric motor 144 is activated so that both slicing disk 140 and stirring rotor 160 rotate. T-valve 164 is set to drain state and suction component 118 activated to provide suction through T-valve 164. The application of suction causes sub-pressure inside device 108 so that atmospheric pressure presses lid 120 downwards, compressing the after-birth. The after-birth encounters parallel knives 138 and is sliced into strips having square cross sections, much like chips. In such a way, it is seen that lid 120 constitutes a translatable disaggregation component having a non-mechanical drive.

As the strips are pressed downwards, slicing disk 140 slices the strips into small bits that fall into connective tissue digester 136 onto filter 152. When lid 120 is substantially at the bottom of organ holder 110 and the after-birth substantially entirely minced, valve 146 is closed and an appropriate amount of digesting liquid is introduced through digesting liquid inlet diaphragm 124 and carried though organ holder 110, past parallel knives 138 and slicing disk 140 by culture medium from wash liquid supply 76 that is heated by liquid heating element 126, cleaning the various components from residue of minced after-birth. When a sufficient amount of culture medium has been introduced into connective tissue digester 136, iris valve 132 is closed and organ holder 110 with lid 120 are discarded.

In connective tissue digester 126, the introduced digesting liquid digests the extracellular matrix and other intercellular connective material of the minced after-birth to yield an organ disaggregate, a process assisted by stirring rotor 160. Liquid, cells and smaller tissue fragments pass through filter 152 to accumulate in lower chamber 150. Tissue fragments that are too large to pass through the perforations in filter 152 are eventually reduced in size by the slicing action of stirring rotor 160 against filter 152.

When the minced after-birth is sufficiently disaggregated, suction component 118 is activated to remove air from and produce a vacuum inside waste separator 162. Valve 146 is opened so that suction is applied to liquid in lower chamber 150 of connective tissue digester 136. Further, positive pressure is applied above the organ disaggregate in connective tissue digester 136 through positive pressure inlet 154 by positive pressure generator 156. The action of stirring rotor 160 on filter 152 prevents filter 152 from being blocked and from tissue remnants remaining thereupon as organ disaggregate is blown out of connective tissue digester 136 and sucked into waste separator 162.

Initially, heavy, solid waste products accumulate in the bottom end of waste separator 162. These waste products are removed by setting T-valve 164 to the drain state and activating suction component 118. When sufficient solid waste product is removed, the organ disaggregate is allowed to rest, leading to a gradual settling of cells at the bottom of waste separator 162 with a liquid supernatant.

When a sufficient proportion of cells has settled, T-valve 164 is set to flow state, allowing cell-rich suspension to flow into organ disaggregate storage container 70. When the cell-rich suspension passes into the organ disaggregate storage container 70, sealing device 96 is activated to seal organ disaggregate storage container 70. A cryopreservative (e.g. 10 percent dimethylsulfoxide with 4 percent human serum albumin in saline) is added to the cell-rich suspension in organ disaggregate storage container 70 through cryopreservative inlet 168 for storage, for example at minus 196 degrees centigrade in the usual way.

Subsequently, device 108 is cleaned and sterilized by the introduction of steam as a sterilizing gas through positive pressure inlet 154. When device 108 is clean and sterile, and additional organ is processed in a similar way.

As described hereinabove, while reducing the present invention to practice the present inventors have uncovered a method of using cells derived from placenta and/or umbilical cord, such as those which can be isolated with the above described device, to generate in-vivo in a host mammal cells/tissues derived from MSCs/HSCs.

In particular, as described in the Examples section which follows, implantation into a host mammal of an implant of unseparated placenta or umbilical cord cells associated with a biocompatible matrix can be used to generate in the host mammal cells/tissues derived from MSCs/HSCs, such as bone, cartilage, adipose tissue and hematopoietic stroma capable of supporting and controlling hematopoiesis.

It will be appreciated that, by virtue of enabling generation in a host mammal of cells/tissues derived from MSCs/HSCs, such as bone, cartilage, adipose tissue and hematopoietic stroma capable of supporting and controlling hematopoiesis, the present invention enables treatment of a disease which is amenable to treatment by administration of such cells/tissues. Diseases which are amenable to treatment by administration of such cells/tissues include those requiring generation/repair of cells/tissues/organs derived from MSCs/HSCs, and/or those requiring therapeutic immune modulation.

As further described hereinbelow, diseases requiring generation/repair of cells/tissues/organs derived from MSCs/HSCs, and whose treatment is enabled by the present invention, include, for example, cartilage/bone damage, myocardial infarct, and myeloablation following cancer treatment; and diseases requiring therapeutic immune modulation include, for example, transplantation-related diseases, tumors/cancers, autoimmune diseases and infectious diseases.

Thus, the present invention provides a method of treating in a subject a disease amenable to treatment by administration of a therapeutic cell population derived from mesenchymal and/or hematopoietic stem cells. The disease treatment method is effected by subjecting cells derived from placenta and/or umbilical cord which are in association with a biocompatible matrix to differentiation-inducing conditions suitable for inducing differentiation of at least some of the placental/umbilical cord cells into the therapeutic cell population.

As used herein, the term “treating” includes curing, alleviating, or stabilizing the disease, or inhibiting future onset or development of the disease.

As used herein, the term “disease” refers to any disease, disorder, condition or to any pathological or undesired condition, state, or syndrome, or to any physical, morphological or physiological abnormality.

As used herein, the term “therapeutic cell population” refers to any population of isolated, aggregated and/or tissue-forming cells which can be derived from placental/umbilical cord cells using suitable differentiation-inducing conditions, and which have a capacity to confer a desired therapeutic effect when transplanted into a subject of the present invention in need of such therapeutic effect.

The treatment method may be employed so as to treat a disease of the present invention in any of various types of organisms. Preferably, the subject is a vertebrate, more preferably a homeotherm, more preferably a mammal, more preferably a eutherian mammal, and most preferably a human.

As is described in the Examples section below, administration of an implant which comprises placental/umbilical cord cells in association with a biocompatible matrix can be used to generate in a mouse bone, cartilage, adipose tissue and hematopoietic stroma capable of supporting and controlling hematopoiesis. Due to the well-established intimate similarities in basic developmental processes shared by mammals, such as those shared by humans and mice, it will be appreciated that the treatment method may also be utilized to generate in a human cells/tissues/organs derived from MSCs/HSCs such as bone, cartilage, adipose tissue and hematopoietic stroma capable of supporting and controlling hematopoiesis. It will be similarly appreciated that a subject of the present invention may belong to any one of various types mammals, in particular any one of various types of eutherian mammals, such as bovines, equines, ovines, canines, felines, and the like.

Thus, according to the present invention there is provided a medical implant for treating a disease of the present invention in a subject of the present invention, where the implant comprises placental/umbilical cord cells of the present invention in association with a biocompatible matrix of the present invention.

The implant may comprise any of various combinations of populations of placental/umbilical cord cells.

Depending on the application and purpose, the implant preferably comprises placental/umbilical MSCs and/or HSCs, more preferably both MSCs and HSCs.

Preferably, the population of placental/umbilical cord cells which comprises MSCs and HSCs is from isolated placenta, umbilical cord and/or trophoblast tissue.

Preferably, the population of placental/umbilical cord cells are unseparated cells derived from placenta, umbilical cord and/or trophoblast tissue.

The capacity of placenta cells to generate a therapeutic cell population of the present invention is shown in Example 1 of the Examples section below.

The capacity of umbilical cells to generate a therapeutic cell population of the present invention is shown in Examples 2-3 of the Examples section below.

The capacity of trophoblast cells to generate a therapeutic cell population of the present invention is shown in Example 5 of the Examples section below.

As is described in the Examples section below, administration of an implant of the present invention which comprises unseparated placental/umbilical cord cells in association with a biocompatible matrix can be used to generate in a mammal cells/tissues which are derived from MSCs/HSCs, such as bone, cartilage, adipose tissue and hematopoietic stroma capable of supporting and controlling hematopoiesis.

Alternately, the placental/umbilical cord cells may be stem cells isolated from placental/umbilical cord perfusate (refer, for example, to Zhang Y. et al., 2004. Chin Med J (Engl). 117:882-887).

The implant may comprise placental/umbilical cord cells derived from any one of various kinds of donors.

Preferably, the placental/umbilical cord cells are derived from a vertebrate, more preferably a homeotherm, more preferably a mammal, more preferably a eutherian mammal, and most preferably a human.

The placental/umbilical cord cells preferably belong to the same species as the subject, and most preferably are syngeneic with the subject, i.e. typically derived from the subject (alternately derived from an identical twin or clone of the subject).

It will be appreciated that placental/umbilical cord cells which are derived from the subject are obtained from the placenta/umbilical cord joining the subject and the mother of the subject during gestation and ejected by the subject's mother during the birth of the subject. Placental/umbilical cord cells which are obtained thusly can be cryopreserved indefinitely and administered according to need to the subject at any time during the lifetime of the subject.

Alternately, the placental/umbilical cord cells may be non-syngeneic with the subject.

Preferably, placental/umbilical cord cells of the present invention which are non-syngeneic with the subject are allogeneic with the subject.

Preferably, the placental/umbilical cord cells which are allogeneic with the subject are haplotype-matched with the subject at one locus, more preferably two loci and most preferably three loci. One of ordinary skill in the art will possess the necessary expertise, depending on the application and purpose, for selecting suitably haplotype-matched placental/umbilical cord cells for practicing the treatment method of the present invention.

Alternately, the placental/umbilical cord cells may be xenogeneic with the subject.

Preferably, placental/umbilical cord cells of the present invention which are xenogeneic with the subject are derived from a placental/umbilical cord cell donor, such as a transgenic pig, which is suitably genetically modified so as to be composed of cells which are minimally immunogenic, i.e. which will avoid triggering hyperacute rejection, when transplanted into a subject such as a human. One of ordinary skill in the art will possess the necessary expertise for selecting a suitably genetically modified xenogeneic placental/umbilical cord cell donor so as to successfully practice the treatment method of the present invention to treat a given subject.

The biocompatible matrix may have any one of various compositional characteristics, depending on the application and purpose.

Preferably, the biocompatible matrix comprises a demineralized matrix of at least one biological tissue, more preferably comprises a demineralized bone matrix (DBM), and most preferably comprises demineralized tooth matrix.

Alternately, the demineralized bone matrix may comprise demineralized skeletal bone matrix.

One of ordinary skill in the art will possess the necessary skill for preparing a suitable biocompatible matrix to enable suitable preparation of an implant of the present invention (refer, for example, to U.S. Pat. Nos. 6,752,831, 6,437,018, 5,510,396, 5,507,813, 5,439,684, 5,314,476, 5,298,254 and 5,284,655).

Preferably, demineralizing biological tissue such as tooth so as to obtain a biocompatible matrix of the present invention is achieved essentially as described in the Examples section below.

The biocompatible matrix may be composed of any of various numbers and combinations of components, each of which having any of various combinations of structural characteristics and/or dimensions.

The biocompatible matrix is preferably composed of particles, more preferably particles having a minimal diameter of about 310 microns and a maximal diameter of about 450 microns.

As used herein the term “about” refers to a variation of plus or minus 10 percent.

One of ordinary skill in the art will possess the necessary skill for a suitable biocompatible matrix of the present invention having desired structural characteristics.

Preferably, biocompatible matrix particles of the present invention having a specific range of diameters are prepared as described in the Examples section which follows.

An implant of the present invention may comprise any one of various numbers of placental/umbilical cord cells per weight or volume of biocompatible matrix.

Preferably, the implant comprises about 1,500,000 of the placental/umbilical cord cells per about 1 milligram of the biocompatible matrix.

As is described in the Examples section below, administration of an implant of the present invention which comprises 1,500,000 placental/umbilical cord cells derived from the placenta/umbilical cord of a syngeneic mammalian subject per 1 milligram of demineralized tooth matrix can be used to generate in the subject cells/tissues which are derived from MSCs/HSCs, such as bone, cartilage, adipose tissue and hematopoietic stroma capable of supporting and controlling hematopoiesis.

Subjecting the placental/umbilical cord cells to the differentiation-inducing conditions may be achieved in-vitro, or in-vivo, using any one of various methods.

Preferably, subjecting the placental/umbilical cord cells to in-vivo differentiation-inducing conditions is effected by administering an implant of the present invention directly to the subject, such that the therapeutic cell population is generated directly in the subject.

Alternately, subjecting the placental/umbilical cord cells to in-vivo differentiation-inducing conditions may be effected by administering the implant to an host so as to generate the therapeutic cell population in the host, after which the therapeutic cell population is removed from the host and suitably administered to the subject.

When subjecting the placental/umbilical cord cells to in-vivo differentiation-inducing conditions by directly administering the implant to the subject, the implant may be administered to an anatomical location of the subject, such as a site of injury, at which localization of the therapeutic cell population and therapeutic effect mediated thereby is required (hereinafter “target location”). Typically, an injured anatomical location will tend to be a good environment for inducing differentiation of stem cells into cells and tissues functioning to repair/heal the injured location.

Alternately, subjecting placental/umbilical cord cells to in-vivo differentiation-inducing conditions may be effected either in the subject or in a host by administering the implant at an ectopic anatomical location which does not correspond to an anatomical location of the subject at which localization of the therapeutic cell population and/or therapeutic effect thereof is desired. The ectopic location may be selected possessing suitable accessibility, morphology differentiation-inducing characteristics, differentiation-permissive characteristics, and/or immunological permissiveness to achieve generation of the therapeutic cell population. Administration of the implant to an ectopic location to achieve differentiation of the therapeutic cell population prior to administration of the latter to a target location may be desirable in circumstances where administration of the implant directly to the target site is expected to be harmful and/or ineffective, for example at a point in time when the target location is in a state of acute inflammation.

As is described and illustrated in the Examples section below, the renal subcapsular location constitutes a highly suitable ectopic location for administration of an implant of the present invention for successfully inducing differentiation of the placental/umbilical cord cells so as to generate a therapeutic cell population of the present invention, such as cells/tissues such as bone, cartilage, adipose tissue and hematopoietic stroma capable of supporting and controlling hematopoiesis.

In order to facilitate in-vivo differentiation of a desired therapeutic cell population from the placental/umbilical cord cells, an implant of the present invention may comprise suitable differentiation factors.

Subjecting the placental/umbilical cord cells to in-vitro differentiation-inducing conditions so as to generate a desired therapeutic cell population may be effected by culturing placental/umbilical cord cells of the present invention in association with a biocompatible matrix in the presence of suitable differentiation factors, in accordance with established prior art teachings (refer, for example, to Zhang Y. et al., 2004. Chin Med J (Engl). 117:882-887).

To induce differentiation of the administered placental/umbilical cord cells into the therapeutic cell population, the implants may be subjected to the differentiation-inducing conditions for any of various durations, depending on the type and extent of differentiation required.

Subjecting the placental/umbilical cord cells to the differentiation-inducing conditions may be effected for a duration selected from a range of about 3 days to about 1,500 days, more preferably about 10 days to about 500 days, and most preferably about 28 days to about 150 days.

As is described and illustrated in the Examples section below, administration of an implant of the present invention to a subject of the present invention can be used to generate in the subject after a duration of 28-150 days cells/tissues which are derived from MSCs/HSCs, such as bone, cartilage, adipose tissue and hematopoietic stroma capable of supporting and controlling hematopoiesis.

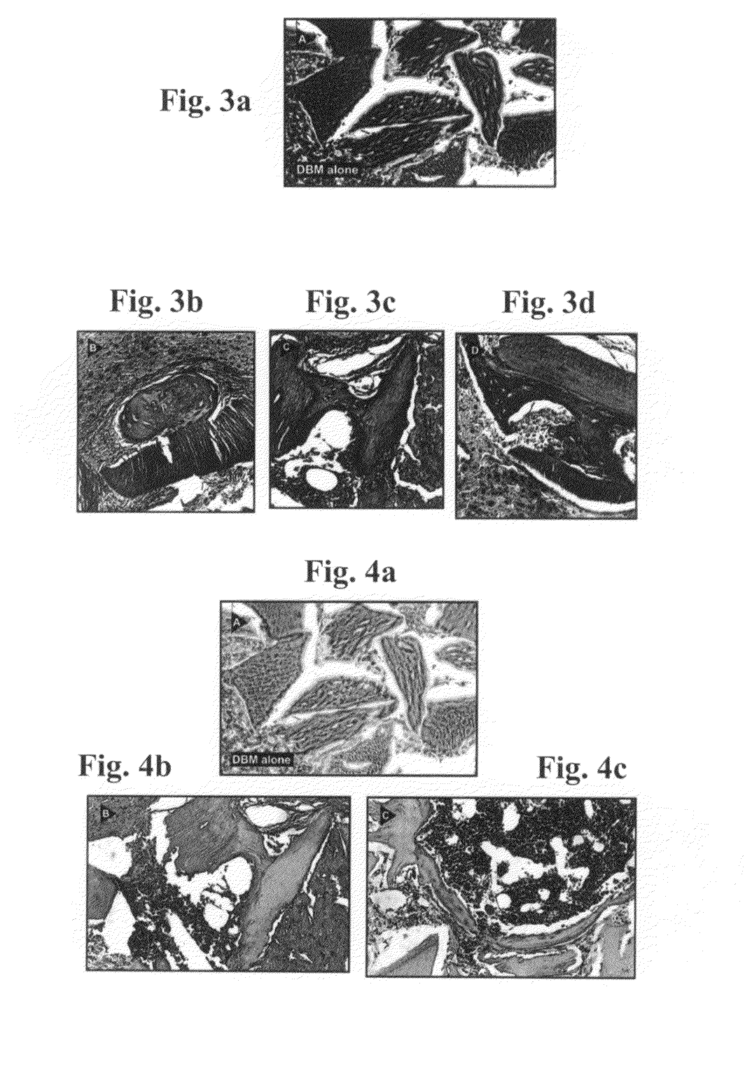

For generation of compact bone, the placental/umbilical cord cells may be subjected to the differentiation-inducing conditions for a duration of at least about 60 days to at least about 150 days. As is described and illustrated in FIGS. 3c-d of the Examples section below, subjecting an implant of the present invention to in-vivo differentiation-inducing conditions for a duration of 60-150 days can be used to generate compact bone.

For generation of hematopoietic tissue, the placental/umbilical cord cells are preferably subjected to the differentiation-inducing conditions for a duration of at least about 60 days to at least about 150 days. As is described and illustrated in FIG. 4b of the Examples section below, subjecting an implant of the present invention to in-vivo differentiation-inducing conditions for a duration of 30 days can be used to generate hematopoietic tissue.

For generation of bone trabeculae with completely developed hematopoietic cavities, the placental/umbilical cord cells are preferably subjected to the differentiation-inducing conditions for a duration of at least about 150 days. As is described and illustrated in FIG. 4c of the Examples section below, subjecting an implant of, the present invention to in-vivo differentiation-inducing conditions for a duration of 150 days can be used to generate bone trabeculae with completely developed hematopoietic cavities.

Depending on the disease to be treated, subjecting placental/umbilical cord cells of the present invention to differentiation-inducing conditions of the present invention may be effected so as to generate any one of various types of therapeutic cell populations suitable for treatment of the disease in accordance with the teachings of the present invention.

Preferably, the differentiation-inducing conditions are selected so as to generate a therapeutic cell population which comprises osteocytes, chondrocytes, adipocytes and/or hematopoietic cells, and/or which forms bone tissue, cartilage tissue, adipose tissue and/or hematopoietic tissue/bone marrow stroma capable of supporting hematopoiesis.

An implant/therapeutic cell population of the present invention may be administered to a subject of the present invention in any one of various ways so as to treat a disease of the present invention.

Ample guidance for administering in accordance with the teachings of the present invention an implant of stem cells in association with a biocompatible matrix so as to treat a disease of the present invention is available in the literature of the art (refer, for example, to: Gurevitch et al., 2003. Stem Cells 21:588-597; and U.S. Pat. Nos. 6,752,831, 6,437,018, 5,510,396, 5,507,813, 5,439,684, 5,314,476, 5,298,254 and 5,284,655).

One of ordinary skill in the art, such as a physician or veterinarian, as appropriate, in particular an artisan specialized in the disease to be treated, will possess the necessary expertise for adapting the teachings of the present invention for suitably treating a particular disease of the present invention in a given subject. In particular, the skilled artisan will possess the necessary expertise for selecting a suitable administration route for suitably formulating/suspending the implant/therapeutic cell population of the present invention, for selecting a suitable dosage for administering the implant/therapeutic cell population, for selecting a suitable regimen for administering the implant/therapeutic cell population, and for suitably monitoring the disease during treatment so as to achieve a desired therapeutic outcome.

Suitable routes of administration of the implant/therapeutic cell population include any of various suitable local and/or systemic routes of administration.

Suitable routes of administration for the implant/therapeutic cell population may, for example, include the intraosseous, intrasynovial, intramuscular, intramyocardial, intracardioventricular, intrahepatic, and intravenous administration routes. Direct injection, with or without surgical exposure of an administration site may be employed, as appropriate. Other administration routes include the oral, buccal, rectal, and topical administration routes.

In order to facilitate administration of the implant/therapeutic cell population to the subject, the implant/therapeutic cell population may be administered concomitantly with physiologically acceptable carriers suitable for the route of administration chosen, disease to be treated, pathological state etc., as appropriate. The physiologically acceptable carrier preferably does not cause significant irritation to an organism and does not abrogate the biological activity and properties of the administered implant/therapeutic cell population.

For injection, the implant/therapeutic cell population may be suspended in an aqueous solutions, preferably in a physiologically compatible buffer such as Hank's solution, Ringer's solution, or physiological salt buffer.

The implant/therapeutic cell population may be intravenously administered to the subject in any one of various ways, e.g. by bolus injection or continuous infusion.

Determination of a therapeutically effective dose of the implant/therapeutic cell population is well within the capability of those skilled in the art, especially in light of the detailed disclosure provided herein, and in light of in-vitro and animal model experiments performed in the art which provide clear guidance for accurately determining therapeutically useful doses in humans.

Depending on the severity of the disease and responsiveness to treatment, dosing of the implant/therapeutic cell population can be of a single or a plurality of administrations until cure is effected or diminution of the disease state is achieved.

An implant of the present invention may be presented in a pack or dispenser device, such as an FDA approved kit, which may contain one or more unit dosage forms containing the implant. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of medical implants, which notice is reflective of approval by the agency of the form of the compositions or human or veterinary administration. Such notice, for example, may be of labeling approved by the U.S. Food and Drug Administration or of an approved product insert.

Thus, the present invention provides an article of manufacture which comprises packaging material and a therapeutically effective dose of an implant of the present invention, where the article of manufacture is identified in print in or on the packaging material for treatment of a disease of the present invention in a subject of the present invention.

As mentioned hereinabove, the treatment method of the present invention may be employed to treat any one of various diseases requiring generation/repair of cells/tissues/organs derived from MSCs/HSCs, and/or those requiring therapeutic immune modulation.

Diseases which require generation/repair of MSC-derived cells/tissues which can be treated using the method of the present invention include, for example, those which require generation/repair of bone (osteogenesis) and/or cartilage (chondrogenesis).

Diseases which require generation of bone/cartilage which can be treated using the method of the present invention include, for example, diseases of osteo-cartilagenous complexes, bone fractures/abnormalities, osteoporosis, cartilage injuries/abnormalities, tooth damage/loss, and the like. For example, the treatment method can be used to generate bone/cartilage for repairing/generating bone/cartilage for maxillofacial or mandibular reconstruction, for repairing injured/damaged intervertebral discs or vertebrae, for repairing/generating joint cartilage (e.g. knee cartilage), as a scaffold for tooth transplants, and for cosmetic treatments.

Diseases which require generation/repair of HSC-derived cells/tissues which can be treated using the treatment method can be used to treat diseases such as, for example, those which require generation/repair of bone marrow stroma, muscle, blood vessels, liver tissue and/or nerve tissue.

For example, the treatment method can be used to treat ischemic heart disease, myocardial necrosis, and heart failure.

Thus, for example, in order to repair ischemic myocardium, an implant of the present invention may be injected adjacent to the ischemic tissue. Similarly, in order to repair/regenerate injured liver tissue, an implant of the present invention may be injected adjacent to the injured site.

The treatment method can be used to generate bone marrow stroma which will support hematopoietic reconstitution following transplantation of autologous or non-syngeneic HSCs in a myeloablatively conditioned or non-myeloablatively conditioned subject. The treatment method can be used to generate bone marrow stroma which will support hematopoiesis in a subject with a hematopoietic system impaired as a result of infection, chemotherapy, and/or irradiation.

Diseases which require immune modulation which can be treated using the treatment method of the present invention include, for example, transplantation-related diseases, tumors/cancers autoimmune and infectious diseases.

Transplantation-related diseases which can be treated using the method of the present invention include, for example, graft rejection and graft-versus-host disease (GVHD). By virtue of providing MSCs/HSCs, which are cells having potent immunosuppressive properties, the treatment method serves to facilitate engraftment of allogeneic or xenogeneic donor-derived grafts, such as cellular, tissue or organ grafts. The treatment method is particularly useful for enabling engraftment of non-syngeneic bone marrow grafts. For similar reasons, by virtue of providing such stem cells, the treatment method can be used to treat GVHD.

The strong immunosuppressive capacity of trophoblast cells and umbilical cord cells is demonstrated in Example 4 of the Examples section, below.

Examples of graft rejection which may be treated using the treatment method include chronic graft rejection, subacute graft rejection, hyperacute graft rejection, and acute graft rejection.

By virtue of providing MSCs/HSCs, which are cells having potent immunosuppressive properties, the treatment method can also be used to treat autoimmune diseases.

General examples of autoimmune diseases which may be treated using the treatment method include a cardiovascular autoimmune disease, a connective tissue autoimmune disease, a gastrointestinal autoimmune disease, a glandular autoimmune disease, a gonadal autoimmune disease, a hematological autoimmune disease, a hepatic autoimmune disease, a mammary autoimmune disease, a muscular autoimmune disease, a neurological autoimmune disease, an ocular autoimmune disease, an oropharyngeal autoimmune disease, a pancreatic autoimmune disease, a pulmonary autoimmune disease, a renal autoimmune disease, a reproductive organ autoimmune disease, a rheumatoid autoimmune disease, a skin autoimmune disease, a systemic autoimmune disease, a thyroid autoimmune disease.

Examples of cardiovascular autoimmune diseases comprise atherosclerosis, myocardial infarction, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing and crescentic glomerulonephritis, antiphospholipid syndrome, antibody-induced heart failure, thrombocytopenic purpura, autoimmune hemolytic anemia, cardiac autoimmunity in Chagas' disease and anti-helper T lymphocyte autoimmunity.

Examples of connective tissue autoimmune diseases comprise ear diseases, autoimmune ear diseases and autoimmune diseases of the inner ear.

Examples of gastrointestinal autoimmune diseases comprise chronic inflammatory intestinal diseases, celiac disease, colitis, ileitis and Crohn's disease.

Examples of glandular autoimmune diseases comprise pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases comprise autoimmune diseases of the pancreas, Type I diabetes, autoimmune thyroid diseases, Graves' disease, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Examples of hepatic autoimmune diseases comprise hepatitis, autoimmune chronic active hepatitis, primary biliary cirrhosis and autoimmune hepatitis.

Examples of muscular autoimmune diseases comprise myositis, autoimmune myositis and primary Sjogren's syndrome and smooth muscle autoimmune disease.

Examples of neurological autoimmune diseases comprise multiple sclerosis, Alzheimer's disease, myasthenia gravis, neuropathies, motor neuropathies, Guillain-Barre syndrome and autoimmune neuropathies, myasthenia, Lambert-Eaton myasthenic syndrome, paraneoplastic neurological diseases, cerebellar atrophy, paraneoplastic cerebellar atrophy and stiff-man syndrome, non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies, dysimmune neuropathies; acquired neuromyotonia, arthrogryposis multiplex congenita, neuritis, optic neuritis and neurodegenerative diseases.

Examples of rheumatoid autoimmune diseases comprise rheumatoid arthritis and ankylosing spondylitis.

Examples of renal autoimmune diseases comprise nephritis and autoimmune interstitial nephritis.

Examples of skin autoimmune diseases comprise autoimmune bullous skin diseases, such as, but not limited to, pemphigus vulgaris, bullous pemphigoid and pemphigus foliaceus, discoid lupus erythematosus.

Examples of systemic autoimmune diseases comprise systemic lupus erythematosus and systemic sclerosis.

By virtue of providing to a subject autologous or allogeneic HSCs which can generate autologous or allogeneic effector cells of the immune system, such as T- and B-lymphocytes, the treatment method can also be used to treat diseases, such as tumors/cancers which are amenable to immunological eradication by such immune effector cells.

General examples of tumors/cancers which may be treated using the treatment method include, but are not limited to, an adenoma, a blastoma, a benign tumor, a bone tumor, a brain tumor, a carcinoma, a cardiovascular tumor, a connective tissue tumor, a gastrointestinal tumor, a glandular tumor, a glioma, a gonadal tumor, a head and neck tumor, a hematological tumor, a hepatic tumor, a lymphoid tumor, a malignant tumor, a mammary tumor, a muscle tumor, a neurological tumor, an ocular tumor, a pancreatic tumor, a precancer, a polyp, a pulmonary tumor, a renal tumor, a reproductive organ tumor, a sarcoma, a skin tumor, a thyroid tumor, and a wart.

Specific examples of tumors/cancers which can be treated using the treatment method include adrenocortical carcinoma, bladder cancer, breast cancer, ductal breast cancer, invasive intraductal breast cancer, breast-ovarian cancer, Burkitt's lymphoma, cervical carcinoma, colorectal adenoma, hereditary nonpolyposis colorectal cancer, colorectal cancer type 1, 2, 3, 6 or 7, dermatofibrosarcoma protuberans, endometrial carcinoma, esophageal cancer, gastric cancer, fibrosarcoma, glioblastoma multiforme, multiple glomus tumors, hepatoblastoma, hepatocellular cancer, acute lymphoblastic leukemia (ALL), acute myeloid leukemia (AML), acute nonlymphocytic leukemia, chronic myeloid leukemia (CML), Li-Fraumeni syndrome, liposarcoma, lung cancer, small cell lung cancer, non-small cell lung cancer, non-Hodgkin's lymphoma, lynch cancer family syndrome II, male germ cell tumor, mast cell leukemia, medullary thyroid carcinoma, medulloblastoma, melanoma, meningioma, multiple endocrine neoplasia, myxosarcoma, neuroblastoma, osteosarcoma, ovarian cancer, serous ovarian cancer, ovarian carcinoma, ovarian sex cord tumors, pancreatic cancer, pancreatic endocrine tumors, familial nonchromaffin paraganglioma, pilomatricoma, pituitary tumor, prostate adenocarcinoma, prostate cancer, renal cell carcinoma, retinoblastoma, rhabdoid tumors, rhabdomyosarcoma, soft tissue sarcoma, head and neck squamous cell carcinoma, T-cell acute lymphoblastic leukemia, teratocarcinoma, uterine cervix carcinoma, Wilms' tumor type 1 or 2, etc.

Virtue of enabling generation of autologous or allogeneic effector cells of the immune system, such as T- and B-lymphocytes, the treatment method can also be used to treat diseases, such as infectious diseases which are amenable to immunological eradication by such immune effector cells. For example, the treatment method can be used to confer resistance to infectious agents such as HIV-1, hepatitis B and hepatitis C and other resistant infectious agents.

Examples of infectious diseases which may be treated by the treatment method include, but are not limited to, a bacterial infection, a fungal infection, a mycoplasma infection, a protozoan infection, and a viral infection.

Thus, the present invention provides a novel device for optimally convenient and effective isolation of placental/umbilical cord cells in a cryostorable format, a novel method of treating essentially any disease amenable to treatment via administration of cells/tissues derived from MSCs/HSCs, and a novel medical implant which can be used for practicing this method.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al. “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### In-Vivo Generation of Cartilage, Bone, Adipose Tissue, and Hematopoietic Stroma by Implantation of Composite Grafts of Placental Cells and Demineralized Bone Matrix: Novel Disease Treatment Method

Introduction: Diseases amenable to treatment via administration of cartilage, bone, adipose tissue, and hematopoietic stroma include a vast number of highly debilitating and/or lethal diseases for which no satisfactory/optimal therapy exists. An optimal strategy for treating such diseases involves administration of mesenchymal and/or hematopoietic stem cells, however, the prior art approaches for practicing such disease treatment involve using adult-stage bone marrow as a tissue source of stem cells, which is associated with various significant disadvantages. For example, isolating mesenchymal and/or hematopoietic stem cells from bone marrow is highly invasive, cumbersome expensive and/or inefficient, and hence essentially impossible to routinely practice according to need. Moreover, the prior art use of adult-stage bone marrow as tissue source of stem cells is further associated with the disadvantage that such adult-stage tissues contain cells having a more limited proliferation/differentiation potential for purposes of regenerative therapy, as well as greater immunogenicity for purposes of donor-to-recipient transplantation, relative to tissue sources at earlier developmental stages. Furthermore, the bone marrow of cancer patients, which often critically require hematopoietic reconstitution via stem cell administration following bone marrow-damaging cancer treatment, is highly unsuitable as a source of stem cells due to contamination, or potential contamination, with malignant cells, even though it theoretically represents an ideal, immunologically matched, stem cell source for such patients. While reducing the present invention to practice, as described below, a method of using placenta as a source of stem cells for treatment of diseases, such as those amenable to treatment via administration of cartilage, bone, adipose tissue, and hematopoietic stroma, was unexpectedly uncovered, thereby overcoming the limitations of the prior art.

Materials and Methods:

Animals: Placentas were obtained from BALB/c mice on the 19th day of pregnancy. Balb/c males at 4 months of age were used as recipients of seeded DMB implants.

Preparation of demineralized bone or tooth matrix (DBM): DBM was prepared as previously described [9]. Incisor teeth or bones obtained from freshly sacrificed mice were cleaned from surrounding soft tissues, placed in a jar and rinsed with magnetic stirring in distilled water for 2-3 hours, in ethanol solutions (70 percent, 96 percent and 100 percent consecutively) for 1 hour, and in diethyl ether for 0.5 hour. The washed tooth/bone was then dried under a laminar flow, pulverized in a mortar with liquid nitrogen and sieved to select for a powder of particles having a diameter of 310-450 microns. The powder obtained was demineralized in 0.6 molar HCl overnight. The demineralized powder was washed to remove the acid, subsequently dehydrated in ethanol and diethyl ether, and then dried. All of the procedures were performed at 4 degrees centigrade to prevent degradation of bone morphogenetic proteins (BMPs) by endogenous proteolytic enzymes.

Preparation of placental cell suspension: Placentas were obtained aseptically from donor mice, mechanically pressed through a stainless steel mesh and suspended in PBS at a cell concentration of 300 million cells per milliliter.

Composition of the graft and experimental protocol: Placental cell-DBM composite grafts were prepared by mixing 10 microliters of cell suspension with 2 milligrams of DBM powder prior to transplantation. Experimental mice were transplanted with the composite graft. Control mice were transplanted with DBM or the placenta cell suspension separately. Representative animals from each group were sacrificed 30, 60 and 150 days following transplantation.

Transplantation protocol: Under general anesthesia an incision was performed above the kidney region and the kidney was temporarily driven out. A small cut was made in the kidney capsule, the composite placental cell-DBM graft was inserted under the capsule using a concave spatula and the kidney was returned in its place. The incision was closed and the skin incision was sealed with stainless steel clips.

Histological evaluation: Tissues obtained at autopsy were fixed in 4 percent neutral-buffered formaldehyde, decalcified, passed through a series of ethanol grades and xylene, and embedded in paraffin. Sections (5-7 microns thick) were stained with picroindigocarmin (PIC) or hematoxylin-eosin stain (H&E) for analysis.

Experimental Results:

A suspension of whole Balb/c placental cells mixed with demineralized bone or tooth matrix (DBM) powder to form a composite graft which was implanted into the renal subcapsular space of recipient Balb/c mice in order to functionally reveal MSCs present in whole placental tissue. DBM is a natural scaffolding and source of bone morphogenetic protein (BMP) normally supporting commitment of MSCs for development of bone-associated stromal cells, i.e. osteocytes, chondrocytes, adipocytes of the bone marrow cavity and cells of bone marrow stromal microenvironment supporting hematopoiesis. The renal subcapsular space was selected as the site of implantation, since it has been previously shown that it does not contain local mesenchymal progenitor cells that could be induced into osteogenesis. At the same time, the renal subcapsular space supplies all the necessary local conditions for supporting the development of an osteo-hematopoietic complex by the transplanted placental cells. Thus, the renal subcapsular space serves as an in-vivo system for investigating the bone-forming capacity of the transplanted placental cells.

Bone formation generated by the composite grafts was analyzed via picroindigocarmin (PIC) histological staining (FIGS. 3a-d), and as early as 30 days following implantation of the grafts, various types of bone-associated tissues originating from placental MSCs were observed (FIG. 3b). New bone and cartilage formation was mostly observed in association with DBM particles. FIGS. 3c-d respectively show newly formed compact bones at 60 and 150 days after implantation.

Hematopoietic tissue generation by the implanted grafts was analyzed via H&E histological staining (FIGS. 4a-c). Hematopoietic tissue generated by the grafts was also seen as early 30 days following implantation (FIG. 4b), when bone marrow cavities were still small, and were also seen at 150 days following implantation when they were big and legibly expressed (FIG. 4c). In addition to compact bone being found in association with DBM particles, entirely remodeled bone trabeculae were seen predominantly associated with hematopoietic cavities (FIG. 4c). Since it is well established that the development, function and long-term maintenance of hematopoietic tissue is crucially dependent on cells of bone marrow stromal origin (known as “hematopoietic microenvironment”), and that such tissue is associated predominantly with bone cavities and trabecular bone, it is apparent that the observed hematopoietic tissue was also produced by placental MSCs present in the implanted grafts.

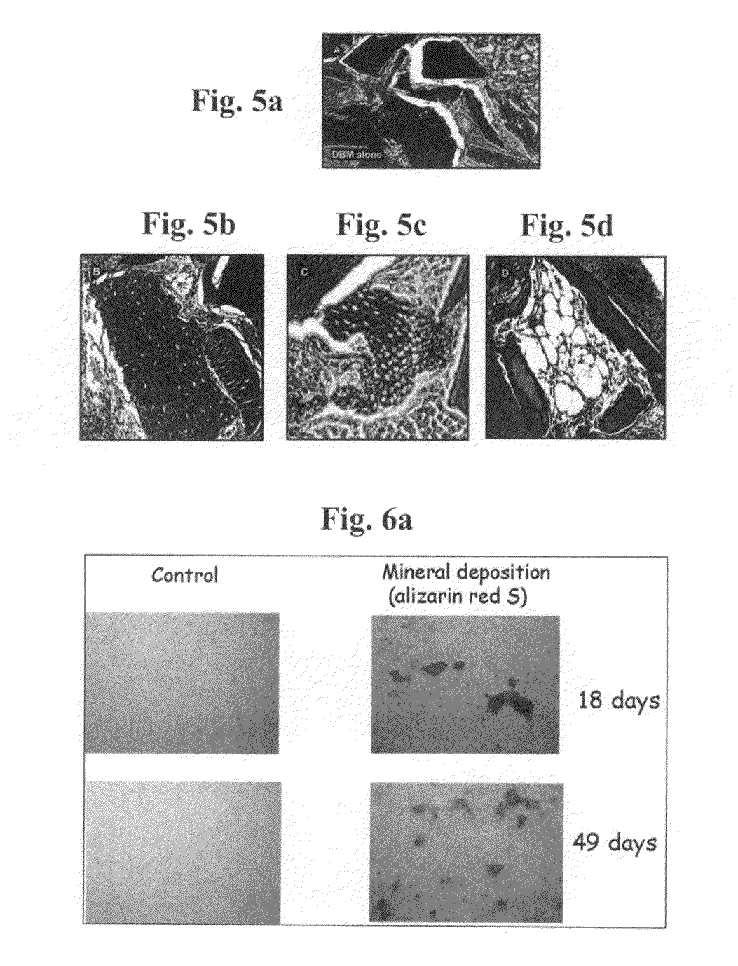

Cartilage and adipose tissue generated by the implants was analyzed via picroindigocarmin (PIC) histological staining (FIGS. 5a-d). Together with osteogenesis, cartilage formation/chondrogenesis generated by the implants was observed at 30 and 150 days following implantation (FIGS. 5b-c, respectively). Moreover, development of adipocytes characteristic for bone marrow cavities (“yellow bone marrow”) occurred adjacent to functionally active hematopoietic tissue (FIG. 5d).

Control implantation of DBM alone, without placental cells, did not lead to development of any of the tissues formed when the composite graft was implanted. Implanted DBM particles remained undegraded at the site of transplantation throughout the whole observation period (FIGS. 3a, 4a and 5a). Likewise, implantation under the kidney capsule of a suspension of placental cells alone never led to any tissue formation (data not shown).

Discussion: There seems to be no question that a huge number of most valuable cells in the placenta including hematopoietic cells and the MSCs that can be preserved indefinitely in liquid nitrogen at minus 196 degrees centigrade can be a source for such cells for each individual secured by cryopreservation of placenta cells instead of throwing such a valuable product to the garbage. The data available suggest that such cells could be used for autologous or allogeneic stem cell transplantation for all indications for the treatment of malignant and non-malignant indications. Furthermore, it has previously been confirmed that a composition consisting of bone marrow cells and DBM could be used for production of bone and cartilage. Ongoing studies suggest that hematopoietic cells can also be used for revascularization of ischemic heart in patients with chronic ischemic heart disease. Recent data associated with stem cell plasticity suggest that such cells may become a source for repair of many other organs including heart muscle, liver cells and neuronal cells for many indications due to acquired or congenital condition. Such cells have a capacity to transform to tissues depending on local physiological conditions with signals to such cells destined for transformation. For example, in order to produce hepatocytes, local hepatic injury must be induced; and in order to transform bone marrow-derived cells into cardiomyocytes, ischemia must be induced, etc.

Considering the fact that life expectancy is constantly prolonged, and considering the fact that each individual might have a high chance to be in need of tissue repair due to traumatic or sport injuries affecting cartilage of muscular skeletal system or degenerative diseases caused by cardiovascular disease affecting the heart or the central nervous system, to mention just a few, it seems reasonable that placenta can serve as a valuable source of uncommitted multipotential source of stem cells to secure many years of future life.

It seems reasonable to assume that a rich source of MSCs and hematopoietic cells from fetal life may have a higher proliferative and differentiation capacity for many future indications as the field of stem cell plasticity and cell biology develops. Considering the fact that the risk of cancer as the age of the population and life expectancy increase, 1 out of 3 individuals is expected to develop a malignant process for which cryopreserved stem cells can serve as a backup in case of need for chemotherapy or use of cells for cell therapy which undoubtedly will become a major clinical indication for treatment of cancer in the future, as is already the case presently.

Considering the fact that MSCs do not express MHC class II which is the most important cell surface determinant that is essential to stimulate the immune system, it seems reasonable to assume that MSCs isolated from placenta may be relatively resistant against rejection thus, suggesting that MSCs isolated from placenta may also support allogeneic recipients in need.

Furthermore, considering the fact that MSCs support hematopoietic cells and considering the fact that engraftment of hematopoietic cells results in transplantation tolerance, cryopreserved placenta cells could serve as a means to induce transplantation tolerance to organ allografts in case of need with the purpose of induction of transplantation tolerance rather than lifelong maintenance immunosuppressive treatment which is mandatory to preserve allografts. In the future, cryopreserved placenta cells may also present a source for facilitation of transplantation tolerance to xenografts based on the capacity of hematopoietic cells to induce transplantation tolerance not only across major histocompatibility barriers but also across species barriers. Thus, cryopreserved placenta cells may serve for induction of unresponsiveness and transplantation tolerance to pancreatic antigens for facilitation of transplantation of pancreatic islets for the treatment of type 1 diabetes.

Similarly, cryopreserved placenta cells may serve for induction of transplantation tolerance to hematopoietic cells with the goal in mind to use such cells to transfer resistance to infectious agents such as HIV-1, hepatitis B and hepatitis C and other resistant infectious agents in the future.

For the purpose of cellular transplantations, many studies in the past have shown that the earlier the source of the graft, the greater the success of transplantation. Considering the fact that placenta cells formed a direct bridge between the fetus and the fully mismatched mother where the rejection is fully avoided spontaneously with no external intervention, and considering the fact that placenta represents the universal mechanism for maintenance of all vertebrates, it seems reasonable to assume that the placenta is the most important biologically conserved organ throughout the ontogeny of all species. Thus, it seems reasonable to assume that placenta cells will have many future uses as a source of cells at earliest stages of development obtainable without the need for intervention during adult life, and will be useful in many potential clinical indications. As such, embryonic cells isolated from placenta, focusing on early MSCs are likely to become potential source of cells for regulation of the immune system in health and disease. Thus, placenta cells could be used for rejuvenation of the immune system and for immunologic and general biologic rejuvenation of various functions.

Being essential for support of multipotential stem cells, the use of MSCs can be foreseen for tissue repair and rejuvenation of malfunctioning organs throughout life, with possible use of MSC infusions for longevity extension by improving the function of different organs as the individual matures and ages.

Considering the newly emerging future indications for stem cell therapy and cell-based therapies based on the use of multipotential stem cells, it is reasonable to assume that many future indications will emerge from research in the field of stem cell biology. There seems to be little doubt to assume that cryopreservation of such cells is of great value since availability of large amounts of cells that can be isolated from the placenta of each newborn represents a unique lifetime opportunity for each newborn which can be made feasible by the right decision by caring parents.

In summary, the present results identify a population of MSCs and hematopoietic stem cells in placenta capable of differentiation into osteocytes, chondrocytes, adipocytes and cells of stromal microenvironment supporting hematopoiesis in vivo. This observation allows consideration of the placenta as a rich source of MSCs and hematopoietic stem cells for every individual who may be in need. Cryopreserved cells isolated from the placenta, which is normally thrown away, may thus represent a revolutionary ready-made source of early autologous and allogeneic pluripotent MSCs with high proliferative and differentiation capacity. These results are in accordance with recent in-vitro investigations of MSCs isolated from human placenta tissue showed their fibroblastoid morphology and capability of being induced in culture into adipocytes and osteocytes (9).

Conclusion: The presently disclosed experimental results teach for the first time that a composite graft composed of a mixture of whole placental cells and demineralized bone matrix can be used to generate osteocytes, chondrocytes, adipocytes and cells of stromal microenvironment supporting hematopoiesis in-vivo in a mammal. As such the presently disclosed results enable optimally convenient treatment of numerous diseases, such as, for example, those requiring generation/repair of mesenchyme-derived tissues, hematopoietic reconstitution, and/or immune-tolerance induction. The disease treatment method enabled by the presently disclosed results, by virtue of employing mesenchymal and/or hematopoietic stem cells derived from the placenta, is clearly superior to prior art methods, since, in sharp contrast, these employ stem cells which must be isolated from bone marrow via a process which is significantly invasive, painful, cumbersome, expensive and/or inefficient.

### Example 2

### In-Vitro Generation of Bone by Cultured Unseparated Umbilical Cord Cells

### Bone Disease Treatment Method

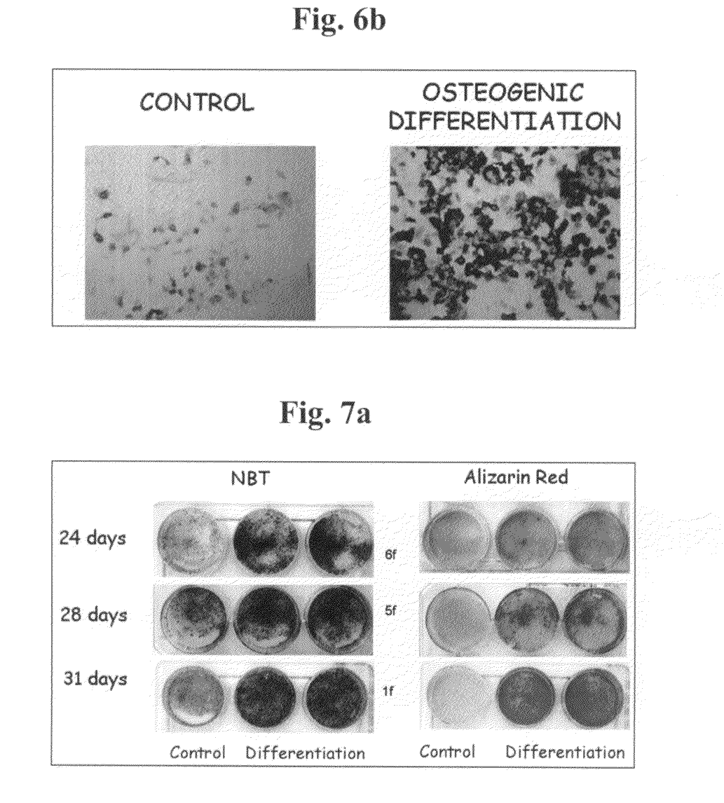

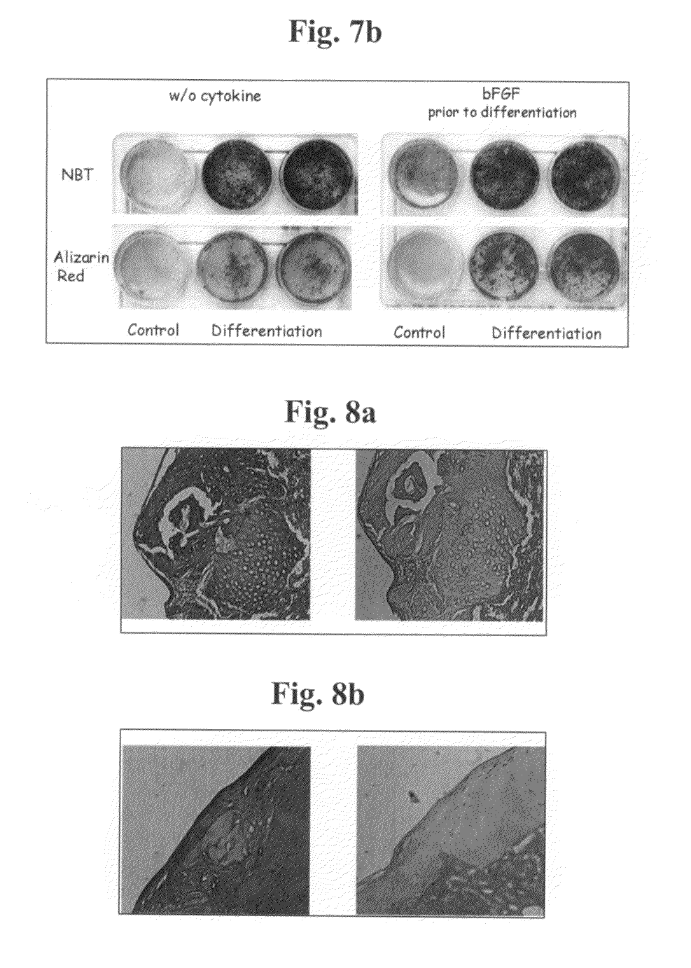

Experiments were conducted to determine whether in-vitro culture of unseparated umbilical cord cells can generate bone in-vitro. Surprisingly, osteogenic differentiation in the cultured cells was observed (FIGS. 6a-b and 7a-b), with the osteogenic differentiation being significantly potentiated by addition of bFGF to the cultures prior to differentiation (FIG. 7b).

### Example 3

### In-Vivo Generation of Cartilage and Bone by Implants of Unseparated Umbilical Cord Cells in Association with Demineralized Bone Matrix

### Bone Disease Treatment Method

Experiments were conducted to determine whether implantation of unseparated umbilical cord cells in association with demineralized bone matrix can generate cartilage and bone in-vitro. Surprisingly, chondrocytic and osteogenic differentiation in the implants were observed after 94 days (FIGS. 8a-b, respectively).

### Example 4

### Inhibition of Mixed Lymphocyte Reaction By Trophoblast and Umbilical Cord Cultured Cells

### Immune Suppression Method

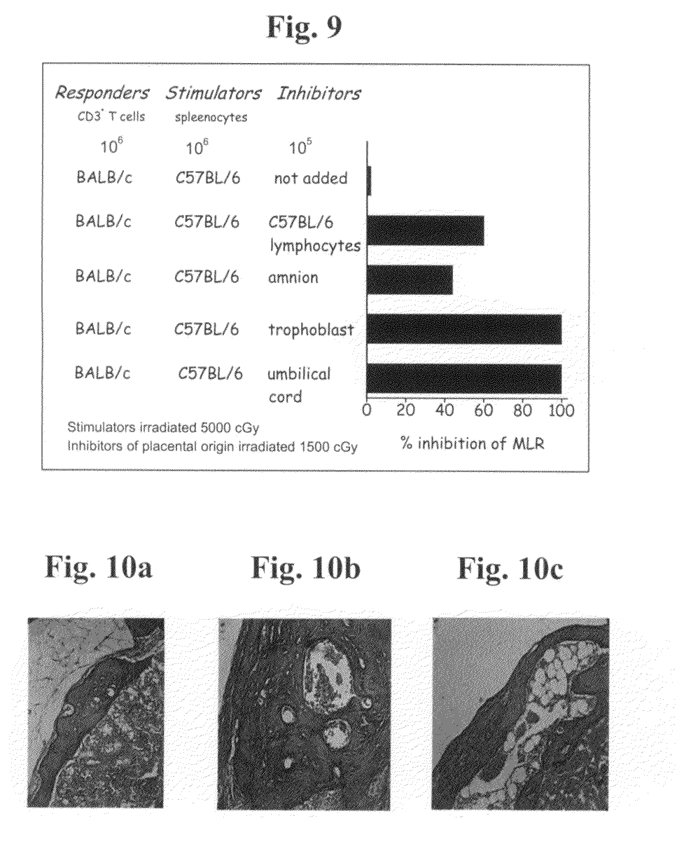

Experiments were conducted to determine whether trophoblast and umbilical cord cultured cells could influence a mixed lymphocyte reaction (MLR) between Balb/c stimulators and C57BL/6 allogeneic responders. Surprisingly, trophoblast and umbilical cord cells were observed to have a very strong immunosuppressive effect on the MLR reaction (FIG. 9).

### Example 5

### Chondrogenesis, Osteogenesis and Trabecular Bone Marrowformation by Unseparated Trophoblast Cells Implanted In-Vivo with Demineralized Bone Matrix

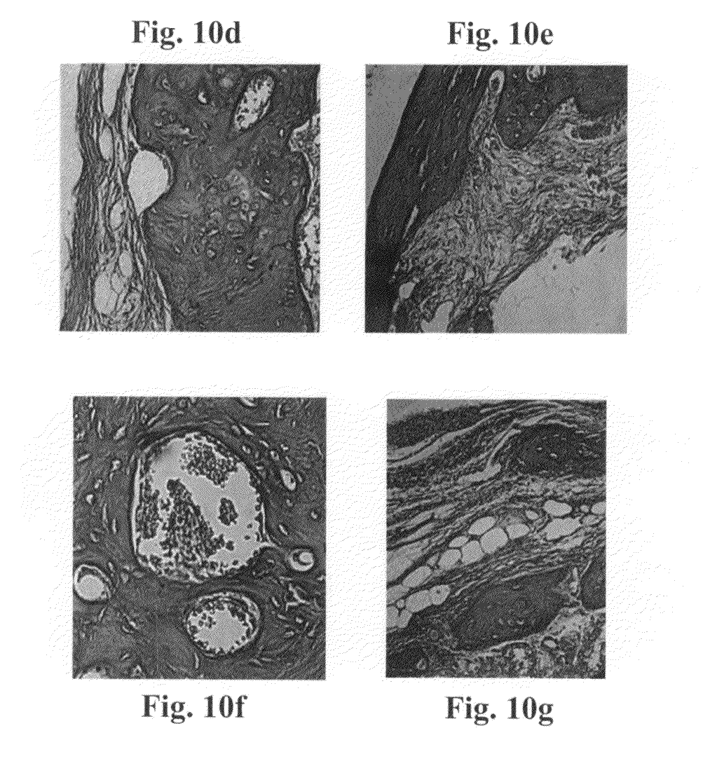

Experiments were conducted to determine whether unseparated trophoblast cells implanted in-vivo with demineralized bone matrix under the renal capsule of a recipient could generate cartilage, bone and/or bone marrow. Surprisingly, the implants were observed to generate hyalin cartilage, primary bone, primary bone with adjacent hematopoietic marrow, and trabecular bone with red and yellow bone marrow (FIGS. 10a-g).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

XXXINSERT BLANK RETURN HERE

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, mentioned in this specification are herein incorporated in their entirety by reference into the specification, to the same extent as if each individual publication, patent, was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

1. Caplan A I. Mesenchymal stem cells. J Orthop Res. 1991; 9:641-650.
2. Prockop DJ. Marrow stromal cells as stem cells for nonhematopoietic tissues. Science. 1997; 276:71-74.
3. Pittenger M F, Mackay A M, Beck S C, et al. Multilineage potential of adult human mesenchymal stem cells. Science. 1999; 284:143-147.
4. Gurevich O, Vexler A, Marx G, et al. Fibrin microbeads for isolating and growing bone marrow-derived progenitor cells capable of forming bone tissue. Tissue Eng. 2002; 8:661-672.
5. Krause D S, Theise N D, Collector M I, et al. Multi-organ, multi-lineage engraftment by a single bone marrow-derived stem cell. Cell. 2001; 105:369-377.
6. Bianchi G, Muraglia A, Daga A, Corte G, Cancedda R, Quarto R. Microenvironment and stem properties of bone marrow-derived mesenchymal cells. Wound Repair Regen. 2001; 9:460-466.
7. Le Blanc K, Rasmusson I, Sundberg B, Gotherstrom C, Hassan M, Uzunel M, Ringden O. Treatment of severe acute graft vs host disease with third party haploidentical mesenchymal stem cells. The Lancet. 2004; 363:1439-1441.
8. Frassoni F, Labopin M, Bacigalupo A, et al. Expanded mesenchymal stem cells (MSC), co-infused with HLA identical hematopoietic stem cell transplants, reduce acute and chronic graft vs host disease: a matched pair analysis. Bone Marrow Transplant. 2002; 29 (2):S2 abstract.
9. Gurevitch OA. The ability of induced osteo-progenitor cells to maintain and rebuild long-term ectopic osteo-hematopoietic foci in vitro. Int J Cell Cloning. 1990; 8:130-137.

1. A method of processing an organ, comprising:
(a) placing an organ in a sealable container;
(b) disrupting the structure of said organ to yield a cell suspension; and
(c) transferring said cell suspension to a sealable cell-suspension storage container, thereby isolating cells of said organ,
wherein said disrupting and said transferring are all performed substantially in a continuous vessel.

2. The method of claim 1, further comprising:
(d) subsequent to (a) and prior to (b), washing said organ.

3. The method of claim 1, further comprising:
(e) prior to (b), contacting said organ with culture medium.

4. The method of claim 1, wherein said disrupting comprises:
(i) physically disrupting said organ to yield organ pieces.

5. The method of claim 1, wherein said disrupting comprises:
(ii) digesting connective tissue of said organ to yield said cell suspension.

6. The method of claim 5, wherein said digesting includes adding an enzyme to said organ.

7. The method of claim 6, further comprising:
(h) adding a cryopreservative to said cell suspension.

8. The method of claim 7, further comprising:
(j) freezing said cell suspension in said sealable cell-suspension storage container.

9. A device for processing an organ comprising:
(a) an aseptic organ disrupter configured to disrupt an organ into a cell suspension; and
(b) a sealable cell-suspension storage container,
wherein said aseptic organ disrupter and said cell-suspension storage container constitute a continuous vessel.

10. The device if claim 9, further comprising an organ washer configured to wash an organ prior to disruption in said organ disrupter.

11. The device of claim 9, further comprising a culture medium inlet functionally associated with said organ disrupter.

12. The device of claim 11, further comprising a culture medium reservoir in fluid communication with said organ disrupter through said culture medium inlet.

13. The device of claim 9, wherein said organ disrupter including a physical organ disrupter.

14. The device of claim 13, wherein said physical organ disrupter includes a disrupter component.

15. The device of claim 14, wherein said disrupter component is rotatable.

16. The device of claim 14, wherein said disrupter component is translatable.

17. The device of claim 14, wherein said disrupter component is vibratable.

18. The device of claim 14, wherein said disrupter component includes a sonic transducer.

19. The device of claim 9, wherein said organ disrupter including a connective tissue digester.

20. The device of claim 19, wherein said connective tissue digester includes a digesting liquid inlet.

21. The device of claim 20, further comprising a digesting liquid reservoir in fluid communication with said connective tissue digester through said digesting liquid inlet.

22. The device of claim 19, further comprising a heater, functionally associated with said connective tissue digester.

23. The device of claim 9, further comprising a solid waste separator to separate solid waste from a cell suspension.

24. The device of claim 9, further comprising a liquid waste separator to separate liquid waste from a cell suspension.

25. The device of claim 9, further comprising an organ holder, substantially a sealable container aseptically reversibly attachable to said organ disrupter.

26. A method of generating a cell population derived from mesenchymal and/or hematopoietic stem cells, the method comprising subjecting to differentiation-inducing conditions cells derived from placenta and/or umbilical cord, said cells derived from placenta and/or umbilical cord being in association with a biocompatible matrix, wherein said differentiation-inducing conditions are selected suitable for inducing differentiation of at least some of said cells derived from placenta and/or umbilical cord into the cell population, thereby generating the cell population.

27. A method of treating in a subject a disease amenable to treatment by administration of a cell population derived from mesenchymal and/or hematopoietic stem cells, the method comprising:
(a) subjecting to differentiation-inducing conditions cells derived from placenta and/or umbilical cord, said cells derived from placenta and/or umbilical cord being in association with a biocompatible matrix, wherein said differentiation-inducing conditions are selected suitable for inducing differentiation of at least some of said cells derived from placenta and/or umbilical cord into the cell population, thereby generating the cell population; and
(b) administering the cell population to the subject, thereby treating the disease in the subject.

28. The method of claim 27, wherein said administering the cell population to the subject is effected by administering to the subject an implant which comprises said cells derived from placenta and/or umbilical cord in association with said biocompatible matrix under a renal capsule of the subject.

29. The method of claim 26, wherein said subjecting said cells derived from placenta and/or umbilical cord to said differentiation-inducing conditions is effected by administering to a host which is not the subject an implant which comprises said cells derived from placenta and/or umbilical cord in association with said biocompatible matrix.

30. The method of claim 26, wherein said subjecting said cells derived from placenta and/or umbilical cord to said differentiation-inducing conditions is effected by implanting under a renal capsule of the subject or of a host which is not the subject an implant which comprises said cells derived from placenta and/or umbilical cord in association with said biocompatible matrix.

31. The method of claim 26, wherein said subjecting said cells derived from placenta and/or umbilical cord to said differentiation-inducing conditions is effected for a duration selected from a range of about 30 days to about 150 days.

32. A method of treating in a subject a disease amenable to treatment by administration of a cell population derived from mesenchymal and/or hematopoietic stem cells, the method comprising administering to the subject an implant which comprises cells derived from placenta and/or umbilical cord in association with a biocompatible matrix, thereby generating the cell population for treating the disease in the subject.

33. The method of claim 32, wherein said administering said implant to the subject is effected by implanting said implant under a renal capsule of the subject.

34. The method of claim 26, wherein the cell population comprises: cells selected from the group consisting of osteocytes, chondrocytes, adipocytes and hematopoietic cells; and/or a tissue selected from the group consisting of bone tissue, cartilage tissue, adipose tissue and hematopoietic tissue.

35. The method of claim 26, wherein said cells derived from placenta and/or umbilical cord are unseparated cells derived from placenta and/or umbilical cord.

36. The method of claim 26, wherein said cells derived from placenta and/or umbilical cord are derived from isolated trophoblast tissue.

37. The method of claim 26, wherein said biocompatible matrix is composed of particles having a minimal diameter of about 310 microns and a maximal diameter of about 450 microns.

38. The method of claim 26, wherein said biocompatible matrix comprises a demineralized matrix of at least one biological tissue.

39. The method of claim 26, wherein said implant comprises about 1,500,000 of said cells derived from placenta and/or umbilical cord per about 1 milligram of said biocompatible matrix.

40. A medical implant for treating in a subject a disease amenable treatment by administration of a cell population derived from mesenchymal and/or hematopoietic stem cells, the implant comprising cells derived from placenta and/or umbilical cord in association with a biocompatible matrix.

41. The medical implant of claim 40, wherein said cells derived from placenta and/or umbilical cord are unseparated cells derived from placenta and/or umbilical cord.

42. The medical implant of claim 40, wherein said cells derived from placenta and/or umbilical cord are derived from isolated trophoblast tissue.

43. The medical implant of claim 40, wherein said biocompatible matrix is composed of particles having a minimal diameter of about 310 microns and a maximal diameter of about 450 microns.

44. The medical implant of claim 40, wherein said biocompatible matrix is a demineralized matrix of at least one biological tissue.

45. The medical implant of claim 40, wherein said implant comprises about 1,500,000 of said cells derived from placenta and/or umbilical cord per about 1 milligram of said biocompatible matrix.

46. The method of claim 27, wherein said subjecting said cells derived from placenta and/or umbilical cord to said differentiation-inducing conditions is effected by administering to a host which is not the subject an implant which comprises said cells derived from placenta and/or umbilical cord in association with said biocompatible matrix.

47. The method of claim 27, wherein said subjecting said cells derived from placenta and/or umbilical cord to said differentiation-inducing conditions is effected by implanting under a renal capsule of the subject or of a host which is not the subject an implant which comprises said cells derived from placenta and/or umbilical cord in association with said biocompatible matrix.

48. The method of claim 27, wherein said subjecting said cells derived from placenta and/or umbilical cord to said differentiation-inducing conditions is effected for a duration selected from a range of about 30 days to about 150 days.

49. The method of claim 27, wherein the cell population comprises:
cells selected from the group consisting of osteocytes, chondrocytes, adipocytes and hematopoietic cells; and/or a tissue selected from the group consisting of bone tissue, cartilage tissue, adipose tissue and hematopoietic tissue.

50. The method of claim 27, wherein said cells derived from placenta and/or umbilical cord are unseparated cells derived from placenta and/or umbilical cord.

51. The method of claim 27, wherein said cells derived from placenta and/or umbilical cord are derived from isolated trophoblast tissue.

52. The method of claim 27, wherein said biocompatible matrix is composed of particles having a minimal diameter of about 310 microns and a maximal diameter of about 450 microns.

53. The method of claim 27, wherein said biocompatible matrix comprises a demineralized matrix of at least one biological tissue.

54. The method of claim 27, wherein said implant comprises about 1,500,000 of said cells derived from placenta and/or umbilical cord per about 1 milligram of said biocompatible matrix.

55. The method of claim 32, wherein the cell population comprises: cells selected from the group consisting of osteocytes, chondrocytes, adipocytes and hematopoietic cells; and/or a tissue selected from the group consisting of bone tissue, cartilage tissue, adipose tissue and hematopoietic tissue.

56. The method of claim 32, wherein said cells derived from placenta and/or umbilical cord are unseparated cells derived from placenta and/or umbilical cord.

57. The method of claim 32, wherein said cells derived from placenta and/or umbilical cord are derived from isolated trophoblast tissue.

58. The method of claim 32, wherein said biocompatible matrix is composed of particles having a minimal diameter of about 310 microns and a maximal diameter of about 450 microns.

59. The method of claim 32, wherein said biocompatible matrix comprises a demineralized matrix of at least one biological tissue.

60. The method of claim 32, wherein said implant comprises about 1,500,000 of said cells derived from placenta and/or umbilical cord per about 1 milligram of said biocompatible matrix.
